(19) United States
(12) Patent Application Publication
(10) Pub. No.: US 2007/0021697 A1
(43) Pub. Date: Jan. 25, 2007

(54) System and method for use of agent in combination with subatmospheric tissue treatment

(75) Inventors: Devin C. Ginther, Converse, TX (US); Archel Ambrosio, San Antonio, TX (US); Teryl B. Sanders, San Antonio, TX (US)

Correspondence Address: LEGAL DEPARTMENT INTELLECTUAL PROPERTY;KINETIC CONCEPTS, INC., P.O. BOX 659508, SAN ANTONIO, TX, 78265-9508, US

(73) Assignee: KCI Licensing, Inc., San Antonio, TX (US)

(21) Appl. No.: 11/494,171

(22) Filed: Jul. 26, 2006

Related U.S. Application Data
(63) Continuation-in-part of application No. 11/189,195, filed on Jul. 26, 2005.
(60) Provisional application No. 60/591,014, filed on Jul. 26, 2004.

(51) Int. Cl. A61F 5/00

(52) U.S. Cl. 602/1

(57) ABSTRACT

A method for uniformly coating a foam or dressing with a polymer-based or metal-based coating incorporating at least one therapeutic or prophylactic agent and a foam or dressing formed by this process. Such foam or dressing is particularly useful in combination with subatmospheric pressure tissue treatment, wherein the foam or dressing formed by the process serves as at least a portion of a screen placed in contact with the tissue and enclosed under a cover. During application of a subatmospheric pressure within the space defined by the cover, the screen compresses and conforms to the tissue, increasing an area of contact between the screen and the tissue. The coating releases the agent directly to the area of contacted tissue. In embodiments where the agent is silver, the coating releases silver ions directly to the contacted tissue to reduce bacterial density thereon.

### CROSS REFERENCE TO RELATED APPLICATIONS

The present application is a continuation-in-part of U.S. patent application Ser. No. 11/189,195 entitled METHOD FOR COATING SUBSTRATE WITH ANTIMICROBIAL AGENT AND PRODUCT FORMED THEREBY filed Jul. 26, 2005, which claims priority to U.S. Provisional Patent Application No. 60/591,014, filed Jul. 26, 2004, assigned to the assignee of the present application, the disclosure of which are incorporated herein by reference.

### BACKGROUND OF THE INVENTION

1. Field of the Invention

This invention relates in general to tissue treatment, and more particularly, but not by way of limitation, to an apparatus and process for the use of therapeutic and prophylactic agents, such as antimicrobial agents, in combination with the application of subatmospheric pressure tissue treatment.

2. Description of the Related Art

Subatmospheric pressure-induced healing of tissue, including but not limited to wounds, has been commercialized by KCI USA, Inc. of San Antonio, Tex., in the form of its “VACUUM ASSISTED CLOSURE®” (or “V.A.C.®”) subatmospheric pressure tissue treatment product line. The subatmospheric pressure-induced healing process in epithelial and subcutaneous tissues was first described in U.S. Pat. Nos. 5,636,643 and 5,645,081 issued to Argenta et al., on Jun. 10, 1997 and Jul. 8, 1997 respectively, the disclosures of which are incorporated by reference as though fully set forth herein. A dressing that was later found to be useful for subatmospheric pressure-induced healing has also been described in commonly assigned U.S. Pat. No. 4,969,880 issued on Nov. 13, 1990 to Zamierowski, as well as its continuations and continuations in part, U.S. Pat. No. 5,100,396, issued on Mar. 31, 1992, U.S. Pat. No. 5,261,893, issued Nov. 16, 1993, and U.S. Pat. No. 5,527,293, issued Jun. 18, 1996, the disclosures of which are incorporated herein by this reference. Further improvements and modifications of such a dressing are also described in U.S. Pat. No. 6,071,267, issued on Jun. 6, 2000 to Zamierowski. Additional improvements have also been described in U.S. Pat. No. 6,142,982, issued on May 13, 1998 to Hunt, et al., and U.S. Pat. No. 7,004,915 issued on February 28, 2006 to Boynton, et al., the disclosures of which are incorporated by reference as though fully set forth herein. Improvements in the use and operation of the connection and conduit components between the dressing and the source of subatmospheric pressure instrumentation have been described in the U.S. provisional patent application Ser. No. 60/765,548, entitled SYSTEMS AND METHODS FOR IMPROVED CONNECTION TO WOUND DRESSINGS IN CONJUNCTION WITH REDUCED PRESSURE WOUND TREATMENT SYSTEMS filed Feb. 6, 2006, disclosure of which is incorporated by reference as though fully set forth herein.

Subatmospheric pressure-induced tissue treatment involves applying subatmospheric pressure to a tissue site over an area, magnitude and time period sufficient to promote healing. In practice, application of the subatmospheric pressure to the tissue site, commercialized by Assignee or its parent under the designations VACUUM ASSISTED CLOSURE® or V.A.C.® therapy, typically involves the mechanical contraction of a wound site with simultaneous removal of excess and interstitial body-liquid. In this manner, V.A.C.® therapy cooperates with the body's natural inflammatory process while alleviating many of the known intrinsic side effects, such as edema caused by increased liquid delivery to the wound site absent the necessary vascular structure for proper removal of waste liquids. As a result, V.A.C.® therapy has been highly successful in the promotion of tissue site closure, healing many sites previously thought largely untreatable.

As is well known in the healing arts, application of certain therapeutic or prophylactic agents to the tissue site may facilitate patient comfort, tissue assessment or directly impact the rate of healing. For example, bacteria may contaminate tissue and interstitial or surface body-liquid at the tissue site. Application of indicating agents known in the art can cause a color change in the presence of a bacterial agent and allow a health care provider to easily and readily ascertain the presence of infection. Further, application of an antimicrobial agent directly to the tissue site may reduce or inhibit bacterial density. Still further, application of anesthetic agents may relieve a patient's discomfort, in those instances where discomfort occurs.

Application of these and other agents directly to the tissue site may be problematic and ineffective for patients receiving subatmospheric pressure tissue treatment. Because the very nature of subatmospheric pressure tissue treatment dictates an atmospherically sealed tissue site, delivery of agents directly to the tissue typically necessitates interrupting application of the subatmospheric pressure, breaking the subatmospheric pressure seal, and disturbing the tissue site. Not only is this a time consuming process for the caregiver, disturbance to the tissue site may increase the possibility of external- and cross-contamination. Disturbance to the tissue site may also cause the patient discomfort in some instances. Furthermore, any applied medicament may be evacuated when the application of subatmospheric pressure tissue treatment is resumed, so a long term, continuous effect from the applied agent may not be realized.

A wide variety of antimicrobial compounds combined with wound dressings can control microbial contamination and potentially lower the rate of infection. Uniform distribution of the antimicrobial can be key to the antimicrobial performance of the wound dressing. What is not known is a reliable method of coating medical wound dressings or foams with a polymer coating agent wherein the entire volume of the dressing is uniformly coated. This occurs for several reasons.

Particularly, some foam dressings are relatively thick, often in the range of about 1.25 inches. The thickness of these dressings limits the coating process, inasmuch as there is no way to insure a uniform coating throughout the entire structure such that the structure is capable of being severed omnidirectionally while still having the desired antimicrobial agent exposed for use in a wound.

Certain coating methods exist, such as vapor deposition (both physical and chemical), electrostatic coating, spraying and sputter coating. However, these coating methods are costly, and are not adaptable to uniformly coating three-dimensional surfaces of certain dressings, such as reticulated foam. In addition, these methods have extensive environmental issues that concern users of the dressings in the medical industry.

Other methods of adding antimicrobials to the dressing, such as additives in the foaming process itself or the use of adjunctive therapies or combination products (e.g. one thin antimicrobial dressing attached to the foam) exist, but are difficult to use and suffer other deficiencies. Particularly, these methods are known to mechanically impact the foam and to materially impact the permeability of the foam.

Because wound sizes and shapes have almost infinite variations, a wound dressing must be adaptable to accommodate the wound and provide appropriate antimicrobial properties to control infection both in the dressing and in the wound. Accordingly, there have been needs to improve dressings for tissue and to develop a process for uniformly coating the dressing or foam with antimicrobial agents sufficient to decontaminate the wound yet simple to use and cost-effective, such that the foam will be adapted for in situ adjustment to match the wound shape and dimension.

### SUMMARY OF THE INVENTION

These and other needs are fulfilled through the development of a process for uniformly coating a foam or other dressing, and a foam or dressing formed by this process with an antimicrobial polymer (i.e., a polymer-based coating incorporating an antimicrobial agent). Such foam or dressing is particularly useful in subatmospheric pressure wound therapy.

Also disclosed are a process for uniformly coating the foam or dressing with a metal, including but not limited to an antimicrobial metal, a foam or dressing formed by this process; its use with a subatmospheric pressure tissue treatment device; and a subatmospheric pressure tissue treatment system and dressing with antimicrobial effects. The foam or dressing formed by the polymer-based and metal-based coating processes discussed herein serves as a screen for use in delivering one or more therapeutic or prophylactic agents, such as antimicrobial agents, to a tissue site in combination with the application of subatmospheric pressure tissue treatment.

In one embodiment, the screen is placed in contact with tissue and a cover is positioned to enclose the screen. The cover also serves to define a space between the cover and the tissue. A pathway is provided between a source of subatmospheric pressure and the space defined by the cover, for application of a subatmospheric pressure within the space defined by the cover. A container is connected to the pathway between the source of subatmospheric pressure and the cover. The container receives the body-liquid drawn along the pathway from within the space defined by the cover.

At least a portion of the screen is a substrate that has been uniformly covered with a coating comprising one or more therapeutic or prophylactic agents. The coating releases at least a portion of the agents within the space defined by the cover. The exterior and interior surfaces of the substrate are covered with the coating to enable the user to expose at least one coated surface of the uniformly covered substrate portion of the screen when adjusting the size and shape of the screen to fit the tissue site.

During application of the subatmospheric pressure within the space defined by the cover, an area of contact between the tissue and the uniformly covered substrate portion of the screen is increased as the tissue microdeforms and the screen compresses and conforms to the surface of the tissue. The coating releases at least a portion of the agents directly to the area of contacted tissue.

In another embodiment, a process for adapting the substrate for treating the tissue during the application of subatmospheric pressure tissue treatment includes the steps of creating a coating solution comprising at least one therapeutic or prophylactic agent; uniformly coating the substrate with the coating comprising the agents, such that an upper surface, a lower surface, side surfaces, and interior surfaces of the screen are uniformly coated; and severing the uniformly coated screen to match the size and shape of the tissue site, such that all exposed surfaces of the screen are uniformly coated sufficient to treat the tissue site during application of the subatmospheric pressure.

The process may further include steps for positioning the screen in contact with the tissue; placing the cover over the screen; providing the pathway between the cover and the source of subatmospheric pressure for applying the subatmospheric pressure within the space defined by the cover; increasing the area of contact between the tissue and the screen by applying the subatmospheric pressure within the space defined by the cover; and releasing at least a portion of the at least one therapeutic or prophylactic agent to the area of contacted tissue.

Many other objects, features and aspects of the present invention will be evident to those of ordinary skill in the relevant arts, especially in light of the foregoing discussions and the following drawings, exemplary detailed description and appended claims.

### BRIEF DESCRIPTION OF THE DRAWINGS

A more complete understanding of the disclosed method and apparatus may be obtained by reference to the following Detailed Description of the Preferred Embodiments, with like reference numerals denoting like elements, when considered in conjunction with the accompanying Drawings, wherein:

FIG. 1 is a flow chart of a process for uniformly coating a wound dressing with antimicrobial agents encapsulated in a polymer-based coating;

FIG. 2 is a schematic diagram of certain steps of the process of FIG. 1;

FIG. 3 is a schematic top plan view of a dressing coated using the process of FIG. 1 or FIG. 19 as applied to a wound site;

FIG. 3A is a schematic top plan view of an alternate embodiment of a dressing coated using the process of FIG. 1 or FIG. 19 as applied to a wound site of FIG. 3;

FIG. 4 is a side view of the dressing of FIG. 3 on a wound site in combination with a subatmospheric pressure therapeutic device;

FIG. 5 is a cross section of the dressing of FIG. 3 taken along line 5-5, illustrating the uniform coating of the dressing;

FIG. 6 is a schematic layout of one embodiment of the apparatus;

FIGS. 7A and 7B are pictorial representations of the housing of the pump and canister for the apparatus of FIG. 6;

FIGS. 8A and 8B are pictorial representations of the apparatus of FIG. 6 supported on a belt and harness respectively;

FIG. 9 is an exploded view of the housing showing the contents of the apparatus of FIG. 6;

FIGS. 10A to 10F show various views of a preferred form of the canister for the apparatus of FIG. 6 and a section of a multi-lumen tube;

FIGS. 11A to 11D show various views of a foam dressing connector for connecting the housing to the dressing;

FIG. 11E is a section of an alternative embodiment of the multi-lumen tube;

FIGS. 12A and 12B show a plan and perspective view of a surgical drape for use with the apparatus of FIG. 6 and FIG. 13;

FIG. 13 is a schematic layout of an alternative embodiment of the apparatus;

FIG. 14A is a perspective view of a fluid sampling port;

FIG. 14B is a perspective view of an alternative embodiment of a fluid sampling port;

FIG. 15A is a perspective view of the back portion of a pump housing for the apparatus of FIG. 13;

FIG. 15B is a perspective view of the front portion of a pump housing for the apparatus of FIG. 13;

FIGS. 16A and 16B are flow charts representing the preferred steps in the implementation of a power management system;

FIG. 17 is a flow chart illustrating the preferred steps in the implementation of pulse therapy;

FIG. 18 is a section view of an alternative embodiment of a cover for use with the apparatus of FIG. 6 and FIG. 13; and

FIG. 19 is a flow chart of a process for uniformly coating a foam or dressing with an antimicrobial metallic coating.

### DETAILED DESCRIPTION OF THE PREFERRED EMBODIMENTS

One embodiment provides a method for uniformly coating a wound dressing with antimicrobial polymers incorporating agents, such as Au, utilizing a process and a wound dressing formed under the process. The method of uniform coating enables a user of the dressing to sever the dressing in any direction and still have all exposed surfaces uniformly coated with the antimicrobial agent sufficient to decontaminate the wound.

An alternative embodiment provides a method for uniformly coating a foam or dressing with a metal-based coating incorporating agents, such as Au, and a dressing formed under the process. As with the polymer-based coating process, the metal-based coating process enables the user to sever the dressing in any direction and still have all exposed surfaces uniformly coated with the agent sufficient to treat the wound.

Silver serves herein as an exemplary agent since the properties of silver allow it to be easily incorporated into both polymer-based coatings and into metal-based coatings. Other agents useful in alternative embodiments include, but are not limited to, therapeutic and prophylactic agents, such as antimicrobial agents, enzymatic debriders, anesthetic agents, chemotherapeutic agents, indicating agents, and growth factors. Antimicrobial agents include but are not limited to antibacterial agents such as antibiotic and bacteriostatic agents. A coating may incorporate single or multiple agents for release to the tissue and to the body-liquid drawn from the tissue. The coating contacts body-liquid and tissue, and releases the agent(s) in the presence of an aqueous environment.

The dressing or screen formed by the coating process is comprised of a substrate uniformly covered with the polymer-based or metal-based coating. The dressing or screen includes a plurality of flow ports or passages provided to allow gas and body-liquid to pass through for facilitating tissue healing. Surfaces of the plurality of ports or passages are also uniformly covered with the coating. The substrate may include, without limitation, material such as foam, yarn, film, filament, fiber, fabric, filler materials, or any combination thereof. The substrate material may be comprised of any substance capable of having the coating applied thereto, including without limitation, nylon, polyester, acrylic, rayon, cotton, polyurethane, other polymeric materials, cellulose materials, such as wood fiber, or any combination thereof. A foam portion of the dressing is preferably of open-celled, reticulated polyurethane, polyether, polyvinylacetate, or polyvinylalcohol construction, but other substitutions or modifications to the foam substrate are considered to be within the scope of this invention.

In one embodiment, a polyurethane foam is uniformly coated with a silver hydrogel polymer. The polymer coating itself contains PVP or Poly(vinyl-pyrrolidone), which is a water-soluble polymer with pyrrolidone side groups, typically used as a food additive, stabilizer, clarifying agent, tableting adjunct and dispersing agent. It is most commonly known as the polymer component of Betadine (a povidone-iodine formulation). In addition, the coating may contain Chitosan, which is a deacetylated derivative of chitin, a polysaccharide that is refined from shells of shrimps, crabs and other crustaceans. Chitosan has also been used in hemostatic dressings. The third optional component of the polymer is preferably Silver Sodium Aluminosilicate, which is silver salt powder with 20% active ionic silver by weight.

In a preferred embodiment, an apparatus and process for treating tissue is provided, wherein the foam or dressing formed by the polymer-based or metal-based coating processes discussed herein serves as a screen for use with a subatmospheric pressure tissue treatment device. The screen is placed in contact with the tissue and enclosed under a generally impermeable cover. The cover provides a substantially air-tight seal over the screen and the tissue, and defines a space over the tissue and under the cover. A liquid conduit is connected between a source of subatmospheric pressure and the cover to provide a pathway for applying a subatmospheric pressure within the space defined by the cover and for drawing interstitial and surface body-liquid therefrom.

When the subatmospheric pressure is applied to the tissue site, the screen compresses and conforms to the surface of the tissue as air is removed from within the space defined by the cover. Microdeformation of the tissue under the cover also occurs. These movements increase an area of contact between the screen and the tissue. In the aqueous environment within the space defined by the cover, the coating releases the agent, such as silver, directly to the increased area of contacted tissue. Increasing the area of contacted tissue brings the coating into direct contact with additional tissue, thereby maximizing the effectiveness of the agent release. In embodiments where the agent is silver, the coating releases silver ions directly to the contacted tissue to help-reduce bacterial density on the area of contacted tissue.

As used herein, references to “wound dressing,” “dressing,” and “foam” as a dressing, are understood to generally refer to the screen comprising the substrate uniformly covered with the coating. In a few instances, the terms have been used to refer to the substrate itself, but their meaning will obvious be to those skilled in the art. The screen is placed substantially over a tissue site to promote the growth of granulation tissue and also to prevent its overgrowth and to release at least one therapeutic or prophylactic agent to the tissue site via the coating. As will be understood by those skilled in the art, the substrate may include, without limitation, material such as foam, yarn, film, filament, fiber, fabric, filler materials, or any combination thereof. The substrate may be comprised of any substance capable of having the coating applied thereto including, without limitation, nylon, polyester, acrylic, rayon, cotton, polyurethane, other polymeric materials, cellulose materials, such as wood fiber, or a combination thereof. Individual fibers are worked (woven, knitted, crocheted, felted, blown, etc.) into a fabric dressing. Foam dressing is preferably of open-celled, reticulated polyurethane, polyvinylalcohol, or polyvinylacetate construction, but other modifications to the foam dressing are considered to be within the scope of this invention.

As used herein, references to “drape” are understood to generally refer to a flexible sheet of construction that is generally body-liquid-impermeable. For purposes of this discussion, use of the term “impermeable” without further qualification, should be understood to generally refer to material and construction that is generally impermeable to body-liquid. Most particular examples include drapes such as those comprising an impermeable elastomeric material, such as a film, the underside of which is at least peripherally covered with a pressure-sensitive adhesive for providing a substantially air-tight seal with a second region of tissue surrounding the tissue site. Alternatively, drapes may be substituted with other covers while still appreciating certain aspects of the invention.

As used herein, references to “subatmospheric pressure” are understood to generally refer to a pressure less than the ambient atmospheric pressure outside the covered tissue site receiving treatment. In most cases, this subatmospheric pressure will be less than the atmospheric pressure at which the patient is located. Subatmospheric pressure tissue treatment may comprise a substantially continuous application of the subatmospheric pressure, where the subatmospheric pressure is relieved only to change the screen, or it can be practiced with the use of a cyclic application of the subatmospheric pressure in alternate periods of application and non-application, or it can be practiced by oscillating the pressure over time.

As used herein, references to “tissue” are understood to generally refer to an aggregation of similar cells or types of cells, together with any associated intercellular materials adapted to perform one or more specific functions including, but not limited to bone tissue, adipose tissue, muscle tissue, dermal tissue, vascular tissue, connective tissue, cartilage, tendons, and ligaments.

As used herein, references to “wound” and “wound site” are understood to generally refer to the tissue site, wherein the term “tissue site” is understood to generally refer to a region of tissue including, but not limited to, a wound or defect located on or within any tissue. The term “tissue site” may further refer to the region of any tissue that is not necessarily wounded or defective, but is instead such as those in which it is desired to add or promote the growth of additional tissue. For example, the subatmospheric pressure tissue treatment may be used in certain tissue regions to grow additional tissue that may be harvested and transplanted to another tissue location.

As used herein, references to “wound fluids,” “wound exudates,” “fluid drainage,” or “fluids” or “liquid” related to the tissue site, are understood to generally refer to body-liquid, wherein the term “body-liquid” is understood to generally refer to any interstitial liquid in the tissues or liquid that has exuded from the tissue or its capillaries.

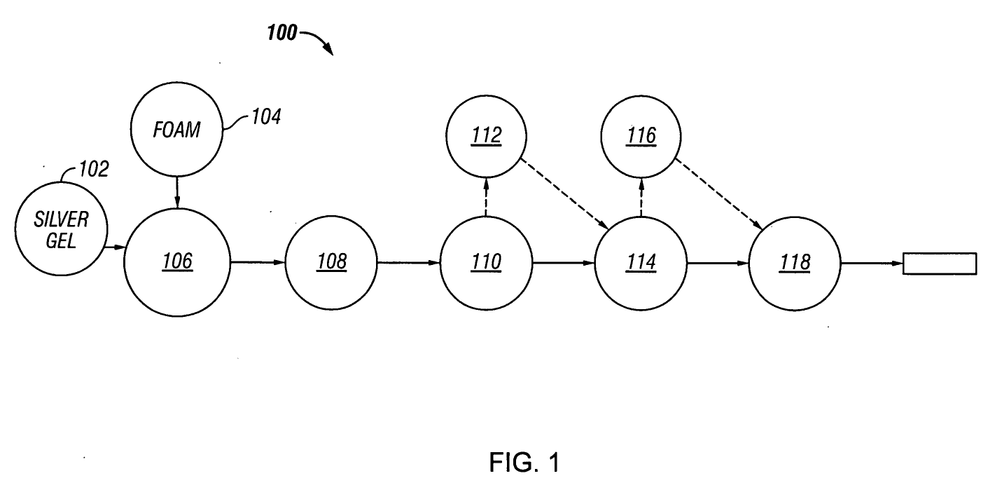

Referring first to FIG. 1, a method 100 for impregnating a foam with a silver polymer coating or antimicrobial coating is shown in the flow chart. First, a hydrophilic gel is combined with silver to create a coating solution, 102. The solution is then placed in a holding tank and continuously agitated in a closed, dark environment, 104. The dark environment is optional, but is included because of the light-sensitivity of silver. In a light-exposed environment, the foam may change color, which results in a non-aesthetic appearance. The foam, which may comprise reticulated polyurethane die-cut foam, is placed in the holding tank, 106. The foam is then saturated with the solution, which is accomplished through soaking or squeezing the foam, 108. Next, excess solution is removed from the foam, 110. Roller nips or similar devices may be utilized to control the amount of solution removed from the foam. Optionally, the weight of the saturated foam, while still wet, may be calculated, 112.

The foam is then placed in a convectional forced-air oven set to a predetermined temperature and time to completely dry the solution-coated foam, 114. Alternatively, to verify the dry condition of the foam, the weight of the foam may be checked again, 116. If light-sensitivity remains an issue, the foam can be packaged in a pouch with a low moisture vapor transmission rate (MVTR), which limits the exposure of the foam to light and to humidity, 118. The foam is now ready for use on such sites as partial thickness burns, traumatic wounds, surgical wounds, dehisced wounds, diabetic wounds, pressure ulcers, leg ulcers, flaps and grafts.

In one example, a foam made by the method described has achieved in-vitro efficacy on two common bacteria—staphylococcus aureus and pseudomonas aeruginosa, with a 20% silver salt load (4% silver by weight, though about 0.1% to about 6% has shown to be at least partially effective). The dressing maintains its effectiveness for 72 hours through a controlled and steady state release of ionic silver. Specifically, a diffusive gradient exists between the silver coating and the anionic rich outside environment that lead to disassociation and eventual transport of the silver ion. Using the above process, over a 6 log reduction or about 99.9999% of pathogenic bacteria have been eliminated between about 24 hours and about 72 hours.

The coating process can easily incorporate other additives, such as enzymatic debriders, anesthetic agents, growth factors and many other biopharmaceuticals. In addition, the coating can be formulated specific to coat thickness, although very thin coatings (about 2 to 10 micrometers) are preferable. The formulation can further be adapted to allow for large particle sizes and different release kinetics, such as concentration and rate and the duration of release.

The coating process can also easily incorporate other additives, singly or in combination. Those skilled in the art can easily adapt this process for polymer-coating other substrates previously listed, such as fiber or film, without undue experimentation.

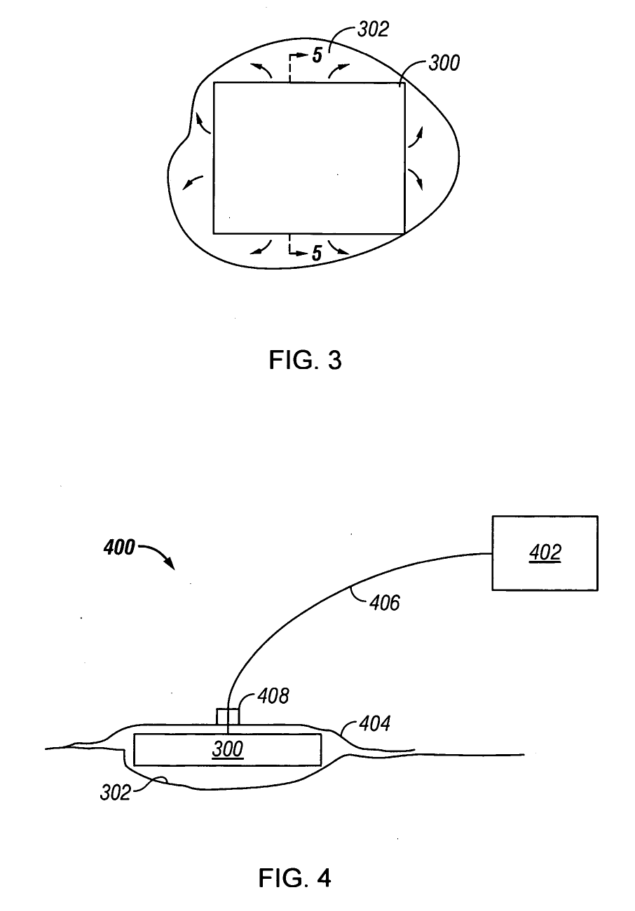

The uniform and impregnated coating allows for delivery of silver ions both outside and within the foam. In this manner, not only is bacteria eliminated on the wound bed, but also within the dressing itself. As discussed below with reference to FIG. 3, this is particularly useful when using the dressing in combination with subatmospheric pressure therapy. Also, odor reduction is an added benefit of this method.

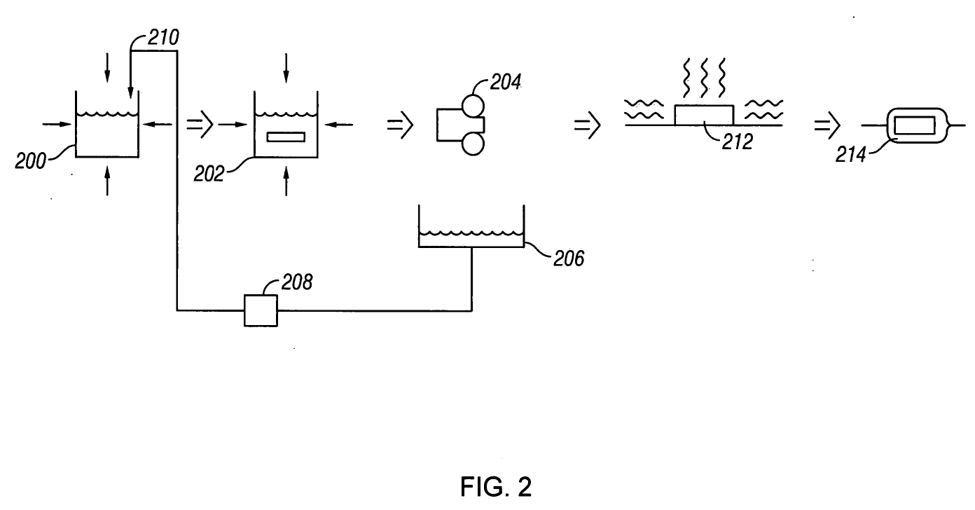

Referring now to FIG. 2, a schematic diagram of certain steps of the process 100 of FIG. 1 is shown. First, the solution of hydrophilic gel and the antimicrobial or other agent, such as silver, is shown in a tank subject to agitation, 200. Next, foam is inserted into the agitating tank, 202. After saturation, the foam is removed and fed through rollers or the like to remove excess solution, 204. The excess solution is captured, 206, and subjected to filtration by a filter sufficiently fine to rid particles from the solution and break apart any chunks of solution that may have formed during the process, 208. A 150-micron filter has been found to be effective during certain silver-solution coating experiments. The filtered solution is then returned to the tank for re-use, 210.

The foam from the removal step 204 is subjected to a convection oven for drying, 212. During certain silver-solution coating experiments, when the temperature of the oven is set at about 90° C., 20 minutes has been found to be an effective drying time. However, it is preferable to dry the foam for about at least 6 minutes to minimize any breakdown of coating. The foam is next packaged in appropriate containers, such as the MVTR pouch or similar containers for shipment to the user, 214.

Referring now to FIG. 3, a schematic top plan view of a dressing 300 coated using the process of FIG. 1 is applied to a wound site 302 as shown. As indicated by the arrows, silver ions from the dressing 300 contact the wound site 302 and effectively eliminate bacteria formed thereon.

The uniform and impregnated coating allows for delivery of silver ions both outside and within the dressing 300. Silver ions release from the uniform coating in the aqueous environment and diffuse to the tissue and into the body-liquid. Pathogens on the tissue, on the underside of the drape, and in the body-liquid that come into contact with the silver ions released from the coating on the outside of the dressing 300 are effectively eliminated. Reduction of bacterial density also occurs as application of the subatmospheric pressure through the dressing 300 effectively pulls body-liquid and accompanying pathogens through the uniformly coated dressing 300, bringing the pathogens into contact with the coating and silver ions within the dressing 300. Additionally, bacterial density within the container is reduced as body-liquid and accompanying silver ions are drawn into the container.

The embodiment of FIG. 3A includes dressing 300′ shown relative to the wound site 320′, and arrows representing silver ions migrating away from the dressing 300′ and contacting the wound site 302′, similar to the dressing 300, the wound site 302, and the arrows of FIG. 3. Whereas the dressing 300 of FIG. 3 has rectilinear edges, in FIG. 3A the edges of the dressing 300′ are adjusted to match the size and shape of the wound site 302′. As used herein, references to “dressing 300,” “pad 300,” and “foam pad 300” are understood to generally refer to the dressing 300′. Similarly, as used herein, references to “wound site 302” are understood to generally refer to the wound site 302′. In practice, the adjusting process is performed by a clinician at the wound site 302′ by severing the edges of a larger-sized dressing, in any direction necessary, to provide a smaller dressing 300′ shaped to match the overall shape of the wound site 302′.

When used in combination with subatmospheric pressure therapeutic devices, such as those commercialized by KCI USA, Inc. (and its affiliates) of San Antonio, Tex. as part of the V.A.C.® product line, the dressing 300 is particularly effective. FIG. 4 is a side view of the dressing 300 of FIG. 3 on a wound site 302 in combination with a subatmospheric pressure therapeutic device 400, which includes a control system 402, a drape 404 for covering the dressing 300 and wound site 302, a subatmospheric pressure hose 406 connected to the control system 402 and to the wound site 302 through the dressing 300, and a connector 408 for connecting the subatmospheric pressure hose 406 to the drape 404. Application of subatmospheric pressure by the control system 402 through the dressing 300 effectively pulls harmful pathogens through the uniformly coated dressing 300, thereby killing the pathogens. In addition, other surfaces of the dressing 300 in contact with the wound site 302 achieve the same result.

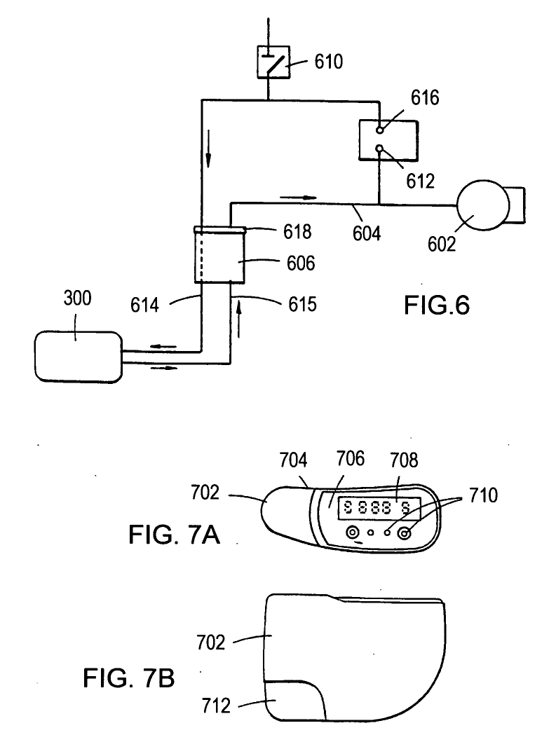

In this embodiment, the subatmospheric pressure therapeutic device 400 preferably serves as the “V.A.C. ATS®” or the “V.A.C. Freedom®” subatmospheric pressure tissue treatment device commercially available from KCI USA, Inc. (and its affiliates) of San Antonio, TX. The “V.A.C. ATS®” device is designed for higher acuity wounds and patients in acute care and long-term care facilities. The V.A.C. ATS® device is illustrated and described below with reference to U.S. Pat. No. 7,004,915, issued to Boynton, et al., and set forth with reference to FIGS. 13 to 17. The “V.A.C. Freedom®” device is a portable subatmospheric pressure tissue treatment device that allows patients to return to daily activities while continuing to receive subatmospheric pressure tissue treatment. The V.A.C. Freedom® device is illustrated and described below with reference to U.S. Pat. No. 6,142,982, issued to Hunt, et al., and set forth with reference to FIGS. 6 to 12B. Suitable alternative subatmospheric pressure therapeutic devices may be the “V.A.C. Instill®” device, the “V.A.C.® Classic” device, the “Mini V.A.C.®” device, or any other “V.A.C.®” model device commercially available from KCI USA, Inc. (and its affiliates) of San Antonio, Tex. Additional suitable alternative devices, dressings and components may be those described in the provisional application previously cited in the Description of the Related Art, the disclosure of which is incorporated by reference as though fully set forth herein. Such alternative V.A.C.® devices, dressings and components also may be generally represented by the subatmospheric pressure therapeutic device 400 and its dressings and components.

Further, in this embodiment the drape 404 serves as a cover, and is preferably the “V.A.C.® Drape” commercially available from KCI USA, Inc. (and its affiliates) of San Antonio, Tex. The subatmospheric pressure hose 406 serves as a liquid conduit, which combined with the connector 408 is preferably the “V.A.C. T.R.A.C.® Pad,” also commercially available from KCI USA, Inc. of San Antonio, Tex.

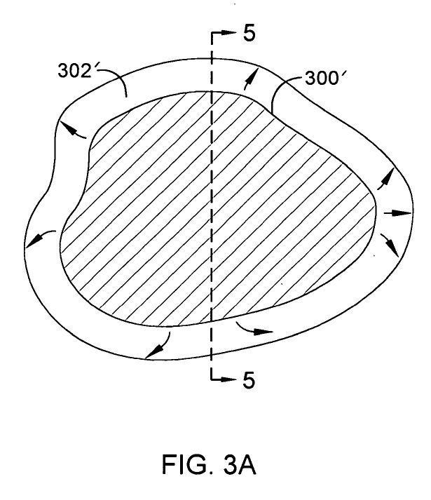

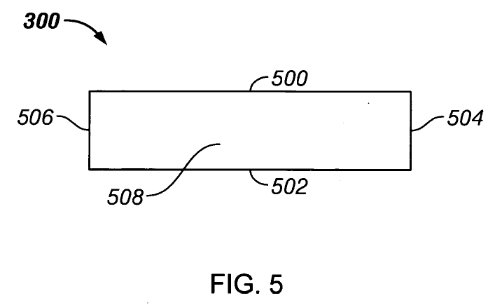

Referring now to FIG. 5, a cross-section of the dressing 300 of FIG. 3 taken along line 5-5 is shown, illustrating the uniform coating of the dressing 300. The dressing 300 has an upper surface 500, a lower surface 502, side surfaces 504, 506 and interior surface 508. All surfaces 500, 502, 504, 506, and 508, are coated with the silver coating, thereby providing an effective barrier to any pathogens that directly contact the surfaces or are indirectly exposed thereto by silver ions migrating away from the dressing 300.

One embodiment of the subatmospheric pressure therapeutic device 400 of FIG. 4 is described in U.S. Pat. No. 6,142,982, issued to Hunt, et al. on May 13, 1998, illustrated and substantially set forth below in FIGS. 6, 7A and 7B, 8A and 8B, 9, 10A to 10F, 11A to 11E, and 12A and 12B, and whose reference is incorporated herein as though fully set forth. A preferred apparatus and process for detecting variations in application of the subatmospheric pressure within the space defined by the cover and for applying intermittent subatmospheric pressure therein is described below with reference to Hunt et al., and clarified with Boynton et al., further below.

Referring to the drawings, the portable therapeutic apparatus comprises a housing 702 (best shown in FIGS. 7A and 7B), having rounded corners and a side 704 which is concavely curved in order to fit comfortably to the wearer's body. The shaping of the housing with curved surfaces is to avoid sharp corners or edges that could dig in to the user or his caregiver. The upper surface 706 is generally flat and has an LCD screen 708 on which details such as applied pressure can be displayed. Control buttons 710 are provided to adjust pressures and treatment intervals. Provision is made for housing a canister within the housing and a snap release cover 712 is arranged for removing or introducing the canister.

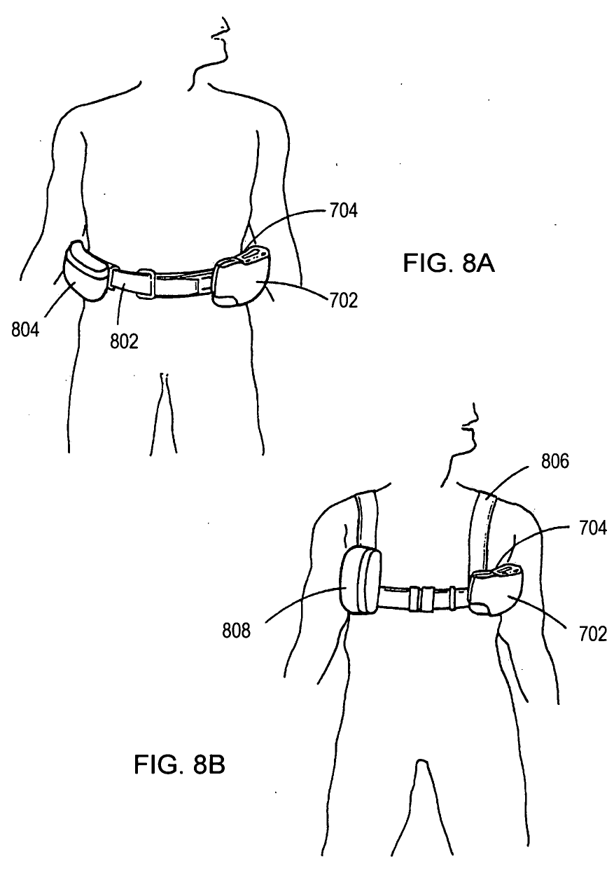

FIGS. 8A and 8B show schematically ways in which the housing 702 may be supported on the patient's body. In FIG. 8A the housing 702 is supported on a belt 802 and its weight is balanced by a similarly rounded casing 804 containing a rechargeable battery pack. FIG. 8B shows an alternative arrangement in which the housing is supported on a harness 806 and again a battery pack is contained in a housing 808, also supported on the harness.

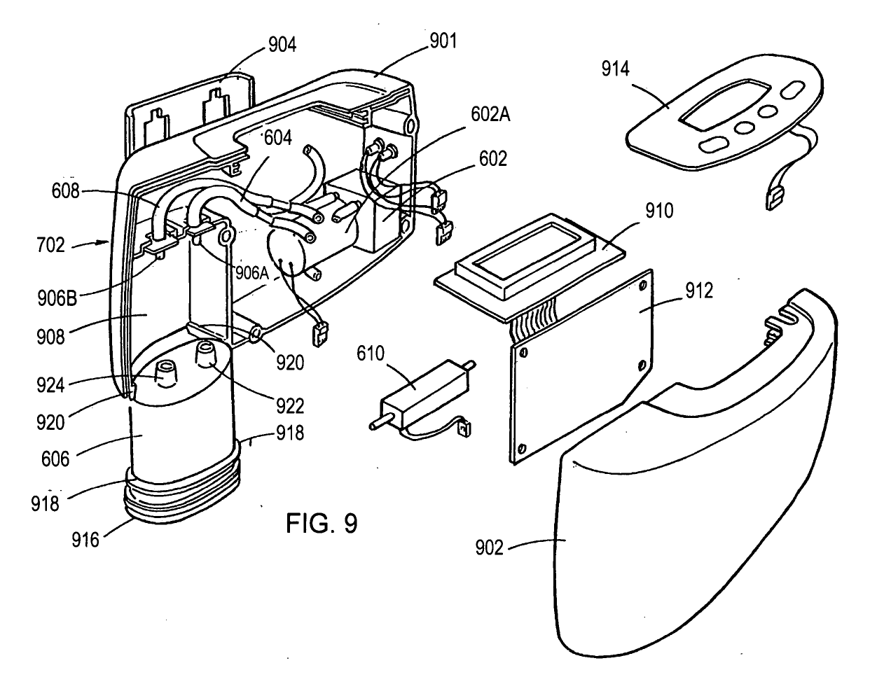

FIG. 9 shows an exploded view of the housing 702 indicating the main components within the housing. The housing consists of front and rear shell moldings 901 and 902 having an external belt clip 904 for attachment to a belt or harness.

Within housing shell 901 is located a subatmospheric pressure pump 602 with associated electric motor 602A and the pump is connected by a silicon rubber tube 604 to a canister spigot 906A in a compartment 908 for the canister 606. Also connected to a second canister spigot 906B via a tube 608 is a pressure relief valve 610 and both tubes 604 and 608 are connected via T-connectors T to pressure transducers (not shown). A microprocessor 910 is mounted on a PCB board 912 and a membrane assembly 914 incorporates an LCD indicator and control buttons.

The apparatus may include means for recording pressures and treatment conditions given to a particular patient which may be printed out subsequently by the physician. Alternatively, the equipment may include a modem and a telephone jack so that the conditions under which the patient has been treated can be interrogated by the physician from a distant station.

Canister 606 is a push fit into the cavity 908 and its lower end is supported in a cover 916. The cover 916 incorporates fingers 918 which are releasably engageable with lips 920 to hold the canister in position. The canister and the latch mechanism is arranged so that when the latch is engaged, the spigots 906A and 906B are in sealing engagement or abutment with tubular protrusions 922 and 924 formed in the top of the canister.

The method of operation of the apparatus can be appreciated from the schematic layout in FIG. 6, in which the canister 606 is connected via tube 615 to a porous dressing 300 at the wound site. Subatmospheric pressure is applied to the wound site via the canister by a tube 604, connected to the pump 602. The pressure in the tube 604 is detected by the transducer 612.

A second tube 614 is connected to the wound site 302 at one end, and also to a pressure relief valve 610 and to a second transducer 616. Tubes 614 and 615 can be combined in a multi-partitioned tube in manner to be described later. By means of tube 614 and transducer 616 the pressure at the wound site can be measured or monitored. A filter 618 is placed at or close to the outlet end of the canister 606 to prevent liquid or solid particles from entering the tube 604. The filter is a bacterial filter which is hydrophobic and preferably also lipophobic. Thus, aqueous and oily liquids will bead on the surface of the filter. During normal use there is sufficient air flow through the filter such that the pressure drop across the filter is not substantial.

As soon as the liquid in the canister reaches a level where the filter is occluded, a much increased subatmospheric pressure occurs in tube 604 and this is detected by transducer 612. Transducer 612 is connected to circuitry which interprets such a pressure change as a filled canister and signals this by means of a message on the LCD and/or buzzer that the canister requires replacement. It may also automatically shut off the working of the pump.

In the event that it is desired to apply intermittent subatmospheric pressure to the wound site, a pressure relief valve 610 enables the pressure at the wound site to be brought to atmospheric pressure rapidly. Thus, if the apparatus is programmed, for example, to relieve pressure at 10 minute intervals, at these intervals valve 610 will open for a specified period, allow the pressure to equalize at the wound site and then close to restore the subatmospheric pressure. It will be appreciated that when constant subatmospheric pressure is being applied to the wound site, valve 610 remains closed and there is no leakage from atmosphere. In this state, it is possible to maintain subatmospheric pressure at the wound site without running the pump continuously, but only from time to time, to maintain a desired level of subatmospheric pressure (i.e. a desired pressure below atmospheric), which is detected by the transducer 612. This saves power and enables the appliance to operate for long periods on its battery power supply.

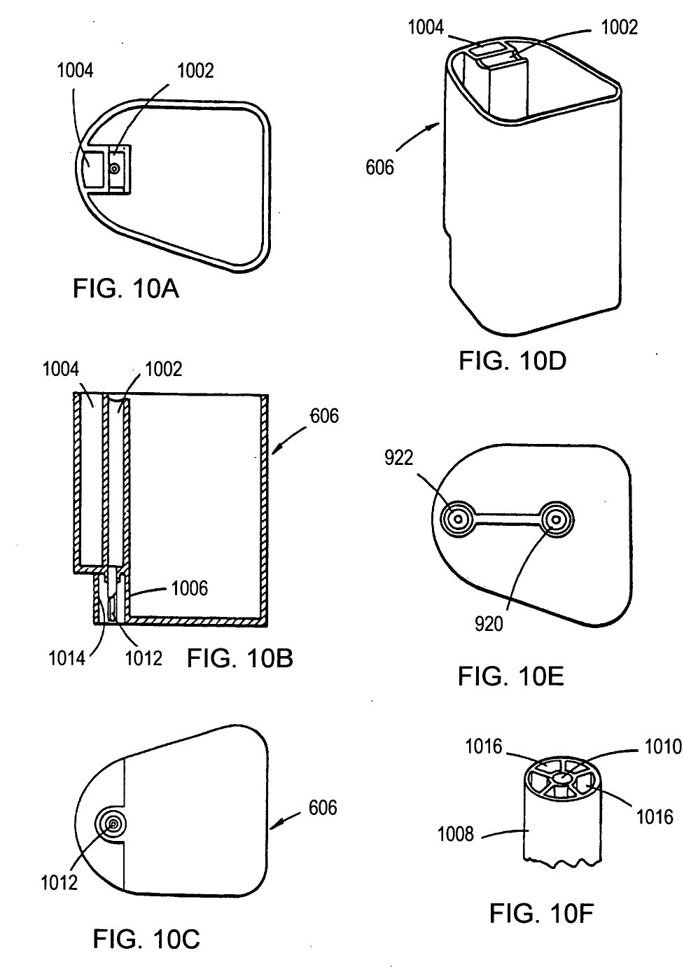

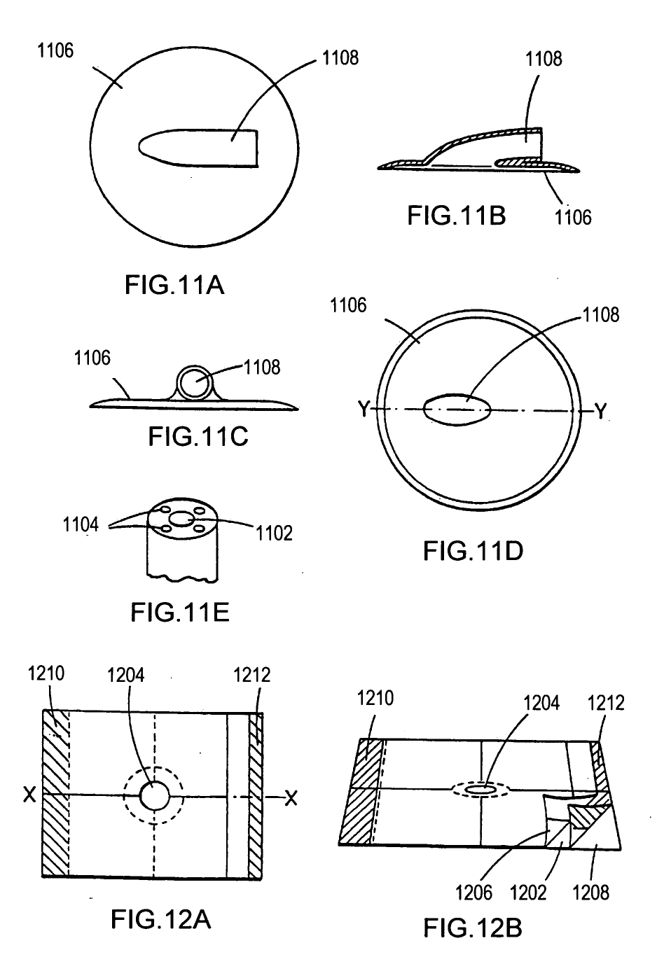

Instead of running two separate tubes to the wound site, it is preferable to contain tubes 614 and 615 in a single tube which is connected through the canister. Thus, for example, tubes 604 and 615 may comprise an internal tube surrounded by an annularg space represented by tube 614. This is illustrated in FIGS. 10A to 10F and in a modified form in FIG. 11E.

In an alternative embodiment, the multi-lumen tube may be constructed as shown in FIG. 11E. In this embodiment, the internal bore 1102 comprises the line 615 (see FIG. 6) and is used to extract fluids from the wound site. Air flow (represented by line 614 in FIG. 6) passes down conduits 1104 located within the walls of the tube. By spacing the conduits 1104 at 90 degree intervals around the tube, the risk of arresting the air flow by kinking or twisting the multi-lumen tube is minimized.

FIG. 10E is a plan view of the top of a preferred shape of the canister, the generally triangular shape in section being chosen to fit better the space within cavity 908 (see FIG. 9). Tubular protrusions on the top of the canister are connected internally of the canister with respectively conduits 1002 and 1004 (see sectional view of FIG. 10B), thus maintaining a separation between the tubes which are represented by lines 604 and 614 in FIG. 6. At the base of the canister, a molding 1006 facilitates connection to a multi-partitioned tube 1008 shown in FIG. 10F. Tube 1008 has a central bore 1010 that is sized to fit over a spigot 1012 in molding 1006. At the same time, the external wall of tube 1008 seals against the inner wall 1014 of molding 1006. Thus, compartment 1002 will connect with central bore 1010 and the compartment 1004 will connect with the annular spaces 1016 of tube 1008. In this way, a conduit 1016 corresponds with line 614 and central bore 1010 with line 615 as shown in FIG. 6.

The partitioned tube need not continue all the way to the wound site 302, but can be connected to a short section of single bore tube close to the wound site.

In the event of an air leak in the dressing at the wound site 302, this can be detected by both transducers 612 and 616 reading insufficient subatmospheric pressure for a specific time period, and then triggering a leak alarm, i.e. a message on the LCD, preferably also with an audible warning.

Typically, the pump 602 is a diaphragm pump but other types of pump and equivalent components to those specifically employed may be substituted.

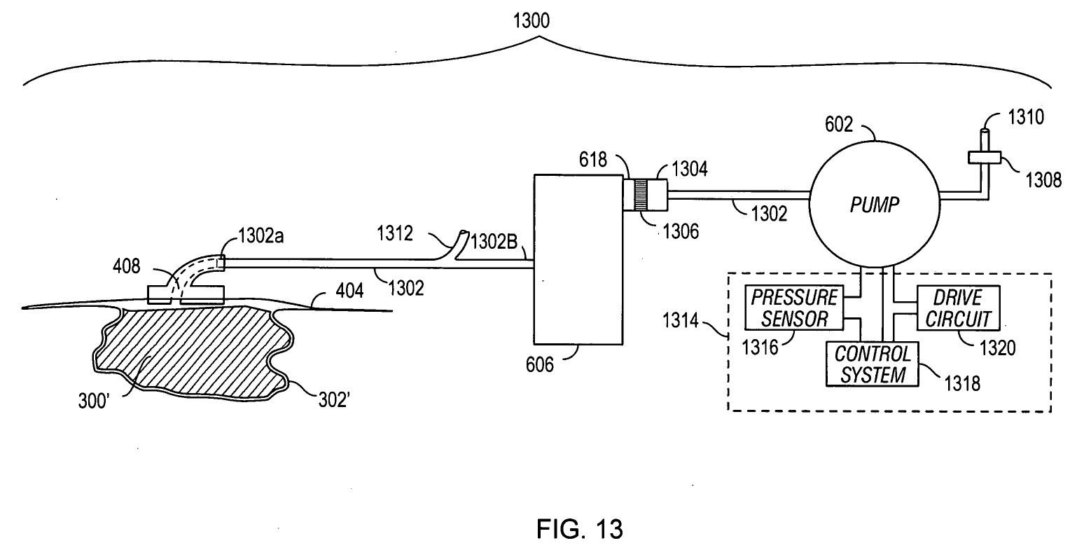

FIGS. 11A-11D show various views of a connector for attaching the multi-lumen tube at the wound site. FIGS. 12A and 12B show a plan and perspective view of a surgical drape for attaching the connector to a porous dressing at the wound site. The connector comprises a molded plastics disc-like cup 1106 having a centrally positioned spout 1108. The spout 1108 is sized to accept, as a closely sliding fit, the end of a multi-lumen tube, e.g. of the kind shown in FIG. 10F or 11E. In use, a porous dressing is cut to correspond with the extent of the wound and pressed onto the wound as shown in FIG. 10 of our PCT application WO 96/05873. Instead of introducing the lumen into the foam dressing, the cup 1106 is pressed onto the porous dressing and secured by a surgical drape. However, if desired, the end of the lumen can be passed into the spout and additionally pressed into the foam. A surgical drape, such as shown in FIGS. 12A and 12B, can be used to secure the connector, lumen and dressing. The drape comprises a polyurethane film 1202 coated on one side with a pressure-sensitive acrylic resin adhesive. A hole 1204 is cut through all layers of the drape and the hole is dimensioned to correspond approximately with the outer cross-section of the spout 1108. Film 1202 has an overall size that allows it to be adhered to the patient's skin around the wound site while, at the same time, securing the connector to the porous dressing. A sufficient overlap around the wound is provided so that an airtight cavity is formed around the wound.

In an alternative form, the drape can be made in two parts, e.g. by cutting along the line X-X in FIG. 12A. With this arrangement, the wound can be sealed by overlapping two pieces of surgical drape so that they overlap each other along a line Y-Y as shown in FIG. 11D.

The surgical drape may include a protective film 1206, e.g. of polyethylene, and a liner 1208 that is stripped off prior to use to expose the pressure-sensitive adhesive layer. The polyurethane film may also include handling bars 1210, 1212, which are not coated with adhesive, to facilitate stretching of the film over the wound site. The dressing is preferably a pad of porous, flexible plastics foam, e.g. reticulated, open intercommunicating cellular flexible polyurethane foam, especially of the kind described in the above-mentioned PCT application WO 96/05873.

Alternatively, a reticulated intercommunicating cellular foam made from flexible polyvinylacetate or polyvinylalcohol foam may be used. The latter is advantageous because it is hydrophilic. Other hydrophilic open celled foams may be used.

In another method of therapy, the foam dressing may be sutured into a wound after surgery and the foam dressing connected to the pump unit by the multi-lumen catheter. Subatmospheric pressure can then be applied continuously or intermittently for a period determined by the surgeon, e.g. from about 6 hours to 4 to 5 days. After this period, the dressing is removed and the wound re-sutured. This therapy improves the rate of granulation and healing of wounds after surgery.

In the foregoing embodiments described with reference to Hunt, et al., the LCD screen 708, microprocessor 910, and PCB board 912 combine to serve as a controller; the subatmospheric pressure pump 602 serves as the source of subatmospheric pressure; the tubes 604 and 615 together serve as the liquid conduit; the transducer 612 serves as the pump pressure transducer; the tubes 608 and 614 together serve as the pressure detection conduit, and the transducer 616 serves as the tissue pressure transducer.

As described above, the tubes 614 and 615 may be contained in one tube to serve as the multi-lumen conduit, wherein the internal bore 1102 serves as a liquid lumen and conduits 1104 serve as pressure detection lumen. Further, the canister 606 serves at the container; the surgical drape serves as the cover; the dressing 300 serves as the screen; and the wound site 302 serves as the tissue site. After the screen is placed in contact with the tissue site, the cover is positioned to enclose the screen, defining the space under the cover and over the tissue site for application of the subatmospheric pressure. It is contemplated that the device may also include wireless communication equipment to allow physicians to remotely access records of the conditions under which the patient has been treated.

An alternative embodiment of the subatmospheric pressure therapeutic device 400 of FIG. 4 is described in U.S. Pat. No. 7,004,915, issued to Boynton, et al., on Feb. 28, 2006, illustrated and substantially set forth below in FIGS. 13, 14A and 14B, 15A and 15B, 16A and 16B, and 17, whose reference is incorporated herein as though fully set forth.

A preferred apparatus and process for detecting whether a container is filled with the body-liquid drawn from within the space defined by the cover, and for preventing the body-liquid from contaminating the source of subatmospheric pressure is set forth below with reference to Boynton et al. A preferred apparatus and process for oscillating application of the subatmospheric pressure over time is also described below with reference to Boynton et al.

The following embodiment is a vacuum assisted system for stimulating the healing of tissue.

Referring now to FIG. 13 in particular, there is illustrated the primary components of a system that operates in accordance with an alternative embodiment. This embodiment 1300 includes a foam pad 300′ for insertion substantially into a wound site 302′ and a wound drape 404 for sealing enclosure of the foam pad 300′ at the wound site 302′. The foam pad 300′ may be comprised of a polyvinyl alcohol (PVA) open cell polymer material, or other similar material having a pore size sufficient to facilitate wound healing. A pore density of greater than 38 pores per linear inch is preferable. A pore density of between 40 pores per linear inch and 50 pores per linear inch is more preferable. A pore density of 45 pores per linear inch is most preferable. Such a pore density translates to a pore size of approximately 400 microns.

Addition of an indicating agent, such as crystal violet, methylene blue, or similar agents known in the art causes a color change in the foam 300′ when in the presence of a bacterial agent. As such, a user or health care provider can easily and readily ascertain if an infection is present at the wound site 302′. It is contemplated that the indicating agent may also be placed in line of the conduit 1302, between the wound site 302′ and the canister 606. In such a configuration (not shown), the presence of bacterial contaminants in the wound site 302′, could be easily and readily ascertained without disturbing the wound bed, as there would be a nearly immediate color change as bacterially infected wound exudates are drawn from the wound site 302′ and through the conduit 1302 during application of subatmospheric pressure.

It is also contemplated that the foam pad 300′ may be coated with a bacteriostatic agent. Addition of such an agent, would serve to limit or reduce the bacterial density present at the wound site 302′. The agent may be coated or bonded to the foam pad 300′ prior to insertion in the wound site, such as during a sterile packaging process. Alternatively, the agent may be injected into the foam pad 300′ after insertion in the wound site 302′.

After insertion into the wound site 302′ and sealing with the wound drape 404, the foam pad 300′ is placed in fluid communication with a subatmospheric pressure source 602 for promotion of wound healing and secondarily, fluid drainage, as known to those of ordinary skill in the art. The subatmospheric pressure source 602 may be a portable electrically powered pump, or other suitable subatmospheric pressure source.

According to one embodiment, the foam pad 300′, wound drape 404, and subatmospheric pressure source 602 are implemented as known in the prior art, with the exception of those modifications detailed further herein.

The foam pad 300′ preferably comprises a highly reticulated, open-cell polyurethane or polyether foam for effective permeability of wound fluids while under subatmospheric pressure. The pad 300′ is preferably placed in fluid communication, via a plastic or like material conduit 1302, with a canister 606 and a subatmospheric pressure source 602. A first hydrophobic membrane filter 618 is interposed between the canister 606 and the subatmospheric pressure source 602, in order to prevent wound exudates from contaminating the subatmospheric pressure source 602. The first filter 618 may also serve as a fill-sensor for canister 606. As fluid contacts the first filter 618, a signal is sent to the subatmospheric pressure source 602, causing it to shut down. The wound drape 404 preferably comprises an elastomeric material at least peripherally covered with a pressure sensitive adhesive for sealing application over the wound site 302′, such that a subatmospheric pressure seal is maintained over the wound site 302′. The conduit 1302 may be placed in fluidic communication with the foam 300′ by means of an appendage 408 that can be adhered to the drape 404.

According to a preferred embodiment, a second hydrophobic filter 1304 is interposed between the first filter 618 and the subatmospheric pressure source 602. The addition of the second filter 1304 is advantageous when the first filter 618 is also used as a fill sensor for the canister 606. In such a situation, the first filter 618 may act as a fill sensor, while the second filter 1304 further inhibits contamination of wound exudates into the subatmospheric pressure source 602. This separation of functions into a safety device and a control (or limiting) device, allows for each device to be independently engineered. An odor vapor filter 1306, which may be a charcoal filter, may be interposed between the first filter 618 and the second filter 1304, in order to counteract the production of malodorous vapors present in the wound exudates. In an alternate embodiment (not shown), the odor vapor filter 1306 may be interposed between the second hydrophobic filter 1306 and the subatmospheric pressure source 602. A second odor filter 1308 may be interposed between the subatmospheric pressure source 602 and an external exhaust port 1310, in order to further reduce the escape of malodorous vapors. A further embodiment allows for first 618 and second filters 1304 to be incorporated as an integral part of the canister 606 to ensure that the filters 618, 1304, at least one of which are likely to become contaminated during normal use, are automatically disposed of in order to reduce the exposure of the system to any contaminants that may be trapped by the filters 618 and 1304.

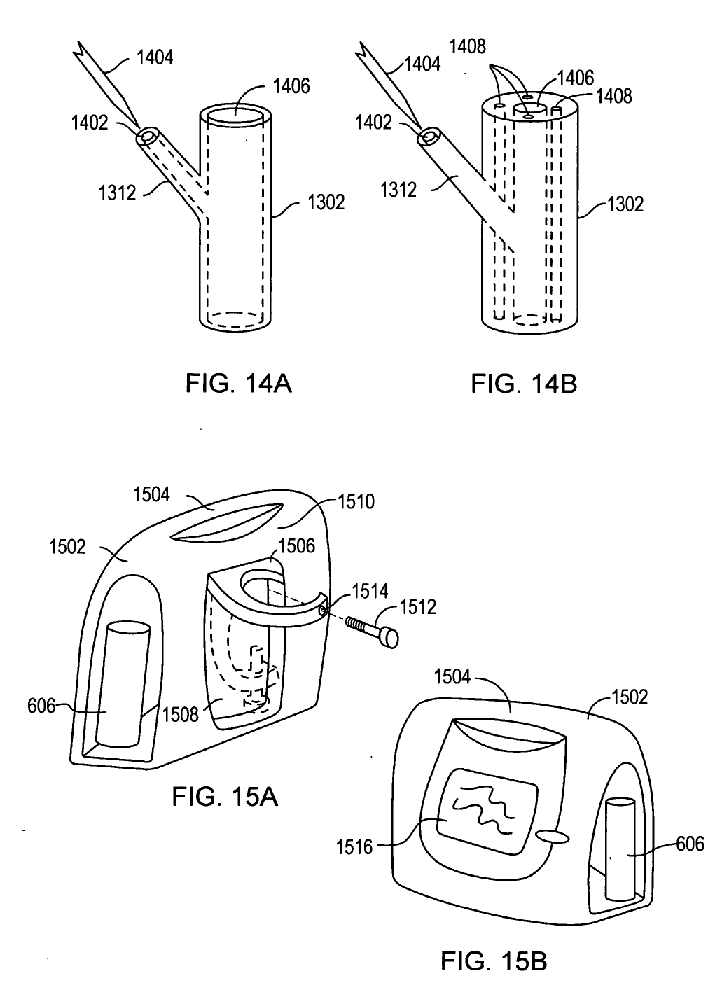

A means for sampling fluids may also be utilized by providing a resealable access port 1312 from the conduit 1302. The port 1312 is positioned between the distal end 1302a of the conduit 1302 and the proximal end 1302b of the conduit 1302. The port 1312, as further detailed in FIGS. 14A and 14B, is utilized to allow for sampling of fluids being drawn from the wound site 302′ by the application of subatmospheric pressure. Although the port 1312 is shown as an appendage protruding from the conduit 1302, it is to be understood that a flush mounted port (not shown) will serve an equivalent purpose. The port 1312 includes a resealable membrane 1402 that after being punctured, such as by a hypodermic needle, the seal is maintained. Various rubber-like materials known in the art for maintaining a seal after puncture can be utilized.

The process by which wound fluids are sampled comprises penetrating the membrane 1402 with a fluid sampler 1404, such as a hypodermic needle or syringe. The sampler 1404 is inserted through the membrane 1402 and into the port 1312 until it is in contact with wound fluids flowing through the inner lumen 1406 of the conduit 1302. As illustrated in FIG. 14B, and further described in U.S. Pat. No. 6,142,982, issued to Hunt, et al. on May 13, 1998, and whose reference is incorporated herein as though fully set forth, the inner lumen 1406 may be surrounded by one or more outer lumens 1408. The outer lumens 1408 may serve as pressure detection conduits for sensing variations in pressure at the wound site 302′. In an alternative embodiment (not shown), the outer lumen or lumens 1408 may act as the subatmospheric pressure conduit, while the inner lumen 1406 may act as the pressure detection conduit. In this embodiment, the fluid sampling port 1312, communicates only with the inner lumen 1406, so as not to interfere with pressure detection that may be conducted by the outer lumens 1408. In an alternate embodiment (not shown) in which the outer lumen 1408 serves as the subatmospheric pressure conduit, the fluid sampling port 1312 communicates with the outer lumen 1408.

The subatmospheric pressure source 602 may consist of a portable pump housed within a housing 1502, as illustrated in FIGS. 15A and 15B. A handle 1504 may be formed or attached to the housing 1502 to allow a user to easily grasp and move the housing 1502.

According to one embodiment, a means for securing the housing 1502 to a stationary object, such as an intravenous fluid support pole for example, is provided in the form of a clamp 1506. The clamp 1506, which may be a G-clamp as known in the art, is retractable, such that when not in use is in a stored position within a recess 1508 of the housing 1502. A hinging mechanism 1510 is provided to allow the clamp 1506 to extend outward from the housing 1502, to up to a 90 degree angle from its stored position. An alternative embodiment (not shown) allows the clamp 1506 to be positioned at up to a 180 degree angle from its stored position. The hinging mechanism 1510 is such that when the clamp 1506 is fully extended, it is locked in position, such that the housing 1502 is suspended by the clamp 1506. A securing device 1512, such as a threaded bolt, penetrates through an aperture 1514 of the clamp 1506, to allow the clamp 1506 to be adjustably secured to various stationary objects of varying thickness.

Alternatively, the securing device 1512, may be comprised of a spring actuated bolt or pin, that is capable of automatically adjusting to various objects, such as intravenous fluid support poles, having varying cross-sectional thicknesses.

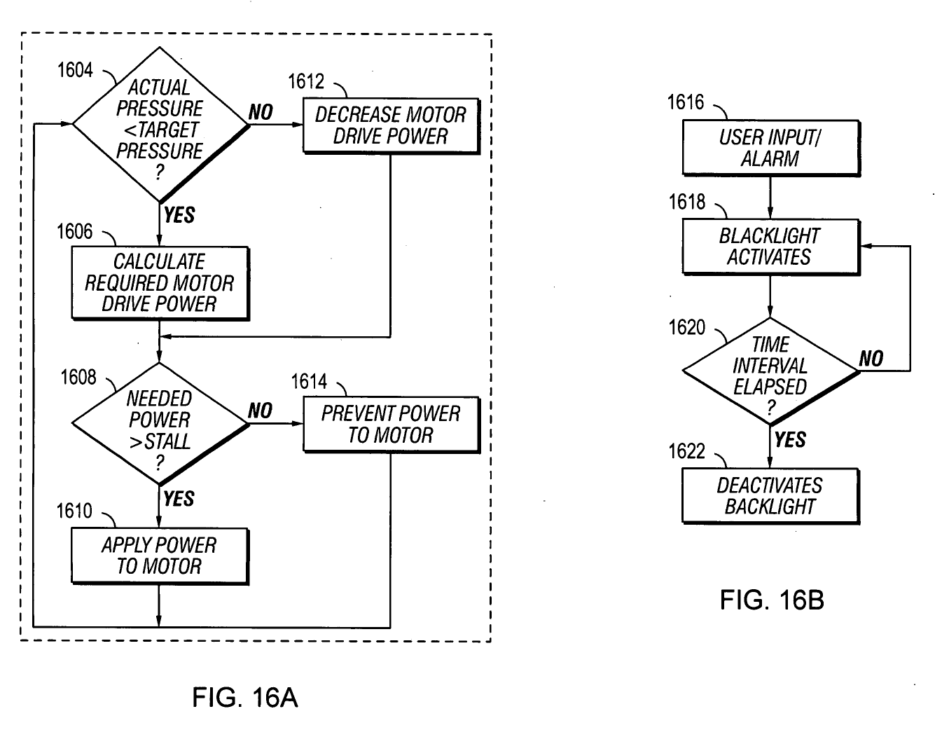

One embodiment also allows for management of a power supply to the subatmospheric pressure source 602, in order to maximize battery life when a direct current is utilized as a power supply. In a preferred embodiment, as illustrated in the flow chart of FIG. 16A, a motor control 1602 determines if the actual pressure is less than or equal to a target pressure 1604. If the actual pressure is less than the target pressure, a tentative motor drive power required to reach the target pressure is calculated 1606. If the tentative motor drive power required to reach the target pressure is greater or equal to the stall power 1608, the tentative motor drive power is actually applied to the motor 1610. If the actual pressure is greater than the target pressure, the tentative motor drive power is decreased and a determination is made as to whether additional power is needed to overcome the stall power 1612. If it is determined that the tentative power is inadequate to overcome the stall power, the tentative power is not supplied to the motor 1614. If the tentative power is adequate to overcome the stall power, the tentative power is actually applied to the motor 1610. The motor control 1602 functions as a closed loop system, such that the actual pressure is continuously measured against the predetermined target pressure. The advantage of such a system is that it prevents power from being supplied to the motor when it is not necessary to maintain the target pressure specified for V.A.C. therapy. Accordingly, battery life is extended because power is not needlessly used to power the motor when it is not necessary.

Battery life is further extended, as illustrated in the flow chart shown in FIG. 16B, by providing a means, such as an integrated software program in a computer processor, for automatically disengaging a backlight of the visual display 1516 of the embodiment 1300 (as seen in FIG. 15B). User input of information 1616, such as target pressure desired, or duration of therapy, activates 1618 a backlight of the visual display 1516 shown in FIG. 15B. User input 1616 may also be simply touching the visual display 1516, which may be a touch activated or a pressure sensitive screen as known in the art. Activation of an alarm 1616 may also activate 1618 the backlight of the display 1516. An alarm may be automatically activated if an air leak is detected at the wound site 302′. Such a leak may be indicated by a drop or reduction in pressure being detected at the wound site 302′. The backlight remains active until a determination is made as to whether a preset time interval has elapsed 1620. If the time interval has not elapsed, the backlight remains active 1618. If the time interval has elapsed, the backlight is automatically extinguished 1622, until such time as the user inputs additional information, or an alarm is sounded 1616.

Referring now back to FIG. 13, battery life is further extended by means of a variable frequency pump drive system 1314, when the pump 602 is an oscillating pump. The pump drive system 1314 consists of a pressure sensor 1316, a control system 1318, and a variable frequency drive circuit 1320. In one embodiment the pressure sensor 1316 measures the pressure across the pump, which is relayed to the control system 1318. The control system 1318 determines the optimum drive frequency for the pump 602 given the pressure measured and relayed by the pressure sensor 1316. The optimum drive frequency for the pump 602 may be determined by the control system 1318 either repeatedly or continuously. The control system 1318 adjusts the variable frequency drive circuit 1320 to drive the pump at the optimum frequency determined by the control system 1318.

The use of the variable frequency pump drive system 1314 allows the pressure of the pump 602 to be maximized. In tests on sample oscillating pumps, the maximum pressure achieved was doubled by varying the drive frequency by only 30%. Additionally, the system 1314 maximizes flow rate over the extended frequency range. As a result, performance of the pump 602 is significantly improved over existing fixed frequency drive system pumps without increasing the pump size or weight. Consequently, battery life is further extended, thus giving the user greater mobility by not having to be tethered to a stationary power source. Alternatively, a similar performance level to the prior art fixed frequency drive system pumps can be achieved with a smaller pump. As a result, patient mobility is improved by improving the portability of the unit.

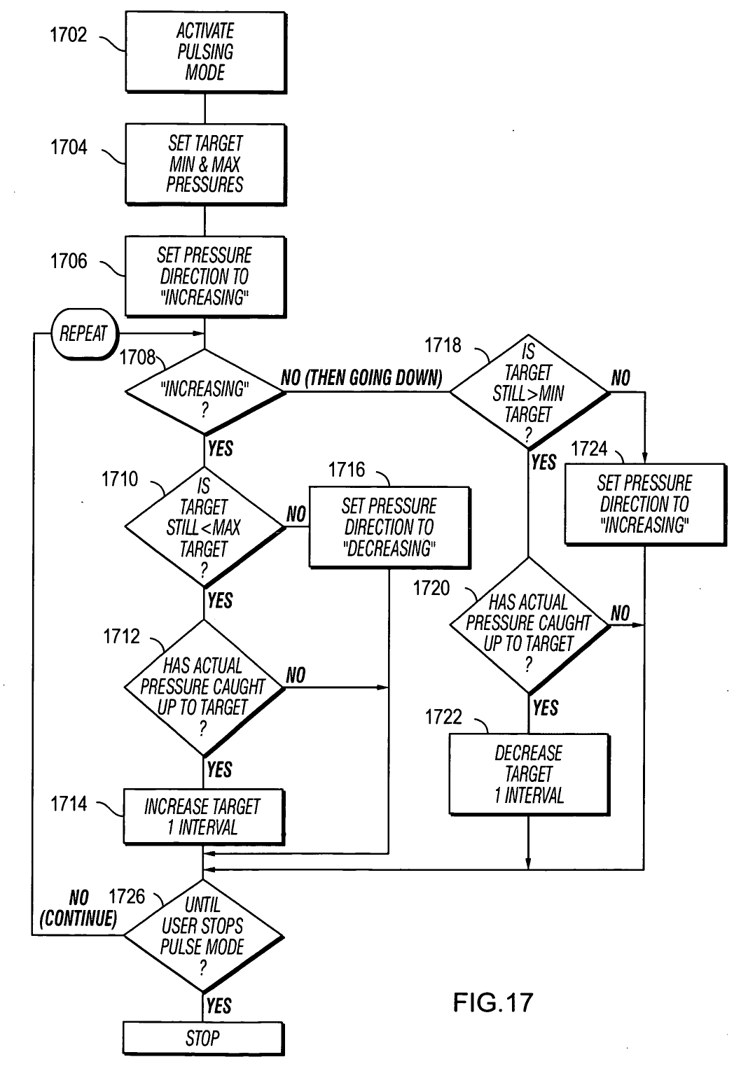

Another embodiment also increases the stimulation of cellular growth by oscillating the pressure over time, as illustrated in the flow chart of FIG. 17. Such an oscillation of pressure is accomplished through a series of algorithms of a software program, utilized in conjunction with a computer processing unit for controlling the function of the subatmospheric pressure source or pump. The program is initialized when a user, such as a health care provider, activates the pulsing mode of the pump 1702. The user then sets a target pressure maximum peak value and a target pressure minimum peak value 1704. The software then initializes the pressure direction to “increasing” 1706. The software then enters a software control loop. In this control loop, the software first determines if the pressure is increasing 1708.

If the actual pressure is increasing in test 1708, a determination is then made as to whether a variable target pressure is still less than the maximum target pressure 1710. If the variable target pressure is still less than the maximum target pressure the software next determines whether the actual pressure has equaled (risen to) the ascending target pressure 1712. If the actual pressure has attained the ascending target pressure, the software increments the variable target pressure by one interval 1714. Otherwise, it refrains from doing so until the actual pressure has equaled the ascending target pressure. If the variable target pressure has reached the maximum target pressure in the test of block 1710 the software sets the pressure direction to “decreasing” 1716 and the variable target pressure begins to move into the downward part of its oscillatory cycle.

The interval may be measured in mmHg or any other common unit of pressure measurement. The magnitude of the interval is preferably in the range of about 1 to 10 mmHg, according to the preference of the user.

If the actual pressure is decreasing in test 1708, a determination is then made as to whether the variable target pressure is still greater than the minimum target pressure 1718. If the variable target pressure is still greater than the minimum target pressure the software next determines whether the actual pressure has attained (fallen to) the descending target pressure 1720. If the actual pressure has equaled the descending target pressure the software decrements the variable target pressure by one interval 1722. Otherwise it refrains from doing so until the actual pressure has equaled the descending target pressure. If the variable target pressure has reached the minimum target pressure in the test of block 1718, the software sets the pressure direction to “increasing” 1724 and the variable target pressure begins to move into the upward part of its oscillatory cycle. This oscillatory process continues until the user de-selects the pulsing mode.

In the foregoing embodiments described with reference to Boynton, et al., the foam pad 300′ serves as the screen; the wound site 302′ serves as the tissue site; the wound drape 404 serves as the cover; the conduit 1302 serves as the liquid conduit; the canister 606 serves as the container; and the electrically powered pump 602 serves as the source of subatmospheric pressure. The appendage 408 serves as the connector interposed between the liquid conduit and the space defined by the cover to secure the liquid conduit to the cover. It is contemplated that the equipment may include wireless communication equipment to allow physicians to remotely access records of the conditions under which the patient has been treated.

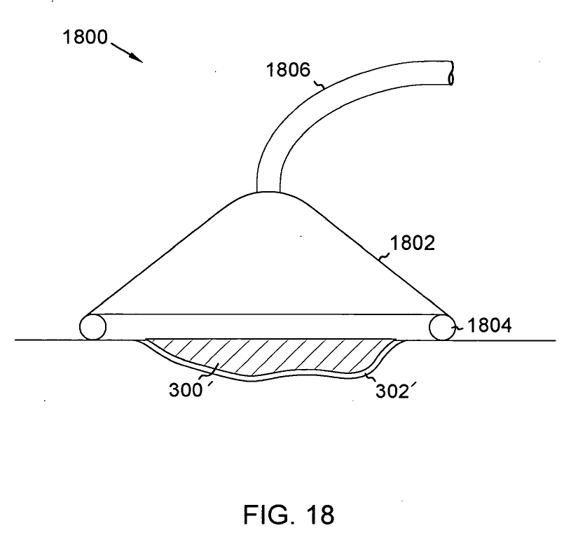

Alternate embodiments of the cover are contemplated including, but not limited to, semi-rigid covers that protect the tissue site 320′. FIG. 18 shows a cup-cuff cover 1800 comprising a semi-rigid cup 1802 and an inflatable cuff 1804. A conduit 1806 is connected to the source of subatmospheric pressure (not shown) and extends through a sealed aperture in the semi-rigid cup 1802. When inflated, the cuff 1804 conforms to the second region of tissue surrounding the tissue site 320′ and is held in place by application of the subatmospheric pressure within the space between the tissue and the cover.

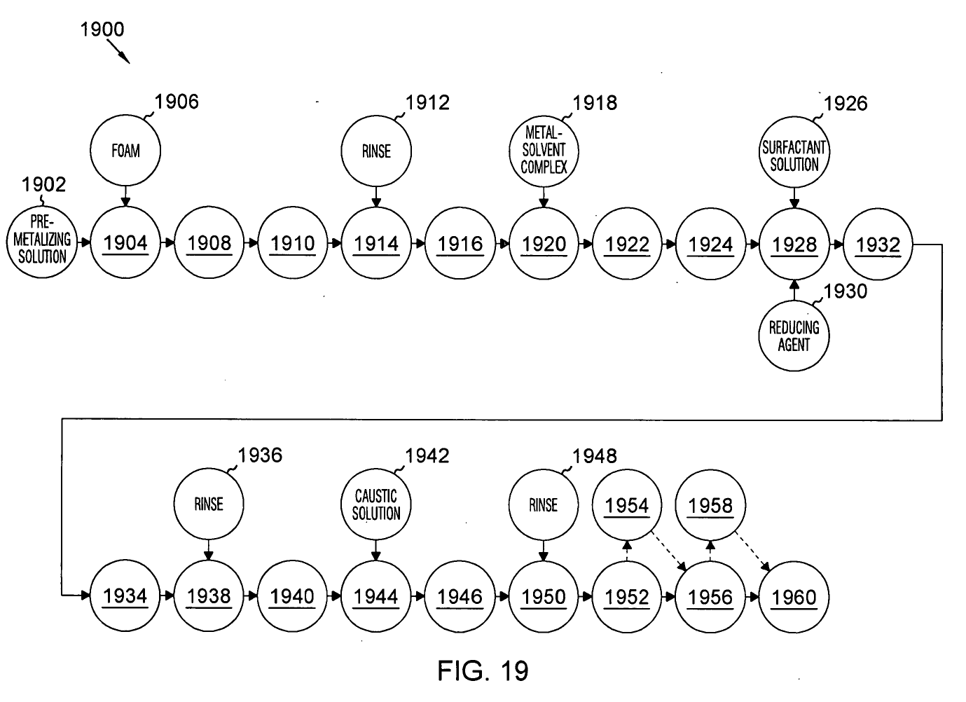

The metallic properties of certain therapeutic or prophylactic agents, such as the antimicrobial silver, also lend themselves to metal-coating the dressing 300. Referring now to FIG. 19, a method 1900 for impregnating foam dressing with the metallic silver coating is shown in the flow chart. First, stannous chloride and muriatic acid are combined to create a pre-metallizing solution, 1902. Any metal salt and/or acid capable of preparing the foam such that the metallic coating better adheres to the surface of the foam may be used in this embodiment. The solution is then placed in a first holding tank and agitated, 1904. The foam, which may comprise reticulated polyurethane die-cut foam, is placed in the first holding tank, 1906. The foam is then saturated with the pre-metallizing solution, which is accomplished through soaking or squeezing the foam, 1908. The foam is removed from the first holding tank and excess pre-metallizing solution is removed from the foam, 1910. Roller nips or similar devices may be utilized to control the amount of solution removed from the foam. A rinse solution is prepared in a second holding tank, 1912. The foam is immersed and thoroughly rinsed, 1914. The foam is removed from the second holding tank and excess rinse is removed from the foam, 1916.

Next, a silver oxide precipitate is combined in a solvent, such as ammonia, to create a silver-solvent complex, 1918. Any solvent capable of dissolving the metal and/or forming a metal-solvent complex may be used. The silver-solvent complex is then placed in a third holding tank and continuously agitated, 1920. The foam is placed in the third holding tank, 1922. The foam is then saturated with the silver-solvent complex, 1924.

Next, a surfactant is completely dissolved in deionized water and placed in a fourth holding tank, 1926. The foam is removed from the third holding tank and placed in the fourth holding tank, 1928. A reducing agent, such as formaldehyde, is added to the surfactant solution and agitated, and the foam is saturated in the solution, 1930. Any reducing agent that is capable of causing the metal to precipitate onto the substrate may be used in this embodiment. The reducing agent precipitates the silver onto the foam to form the metal-coated foam, 1932. The foam is removed from the fourth holding tank and excess solution is removed from the foam, 1934. A rinse solution is prepared in a fifth holding tank, 1936. The foam is immersed and thoroughly rinsed, 1938. The foam is removed from the fifth holding tank and excess rinse is removed from the foam, 1940.

Next, a mild caustic soda solution is prepared and placed in a sixth holding tank, 1942. The foam is immersed in the sixth holding tank and saturated in the caustic soda solution, 1944. The foam is removed from the sixth holding tank and excess caustic solution is removed from the foam, 1946. A rinse solution is prepared in a seventh holding tank, 1948. The foam is immersed and thoroughly rinsed, 1950. Next, the foam is removed from the seventh holding tank and excess rinse is removed from the foam, 1952. Optionally, the weight of the saturated foam, while still wet, may be calculated, 1954.

The foam is then placed in a convectional forced-air oven set to a predetermined temperature and time to completely dry the metal-coated foam, 1956. Alternatively, to verify the dry condition of the foam, the weight of the foam may be checked again, 1958. The foam is then packaged in a moisture vapor transmission rate pouch, if preferred, 1960. The foam is now ready for use on the tissue site, which may include without limitation, any site that may benefit from subatmospheric pressure tissue treatment, such as partial thickness burns, traumatic wounds, surgical wounds, dehisced wounds, diabetic wounds, pressure ulcers, leg ulcers, flaps and grafts.

It is understood that the foregoing coating-process steps, components, component proportions, the amount of time the substrate is immersed in the solutions, and the process for applying the solution to the substrate may vary to accommodate the substrate material and the agent to be coated on the substrate. Such variations are considered to be within the scope of this invention. Those skilled in the art can easily adapt the foregoing coating process for metal-coating other substrates, such as fiber or film, without undue experimentation.

A preferred embodiment uses a metallic coating process provided by Noble Fibers Technologies, Inc., of Clarks Summit, Pa., for producing the “V.A.C. GranuFoam® Silver” antimicrobial silver-coated foam dressing product commercialized by KCI USA, Inc. (and its affiliates) of San Antonio, Tex., for use in combination with their V.A.C.® subatmospheric pressure tissue treatment devices. Although portions of the metallic coating process used by Noble Fibers are proprietary and may not be publicly known, similar techniques will be known to those skilled in the art without undue experimentation.

The V.A.C. GranuFoam® Silver antimicrobial silver-coated foam dressing has achieved in-vitro efficacy on two common bacteria-staphylococcus aureus and pseudomonas aeruginosa, with a uniformly coated 99.9% pure silver metallic coating (4-12% silver by weight, though as little as 0.1% has shown to be at least partially effective). The coating is approximately 1-3 micrometers thick. The dressing maintains its effectiveness for at least 72 hours through a controlled and steady state release of ionic silver, providing over a 4 log reduction or about 99.99% of pathogenic bacteria have been eliminated between about 24 hours and about 72 hours. The coated dressing maintains the physical properties of the foam dressing substrate, which allows for direct and complete contact with the tissue site under application of the subatmospheric pressure.

An alternate embodiment includes uniformly coating a fiber substrate with a metallic agent, such as silver, wherein all fibers are circumferentially covered with the metallic coating. In this embodiment, the fiber is worked (woven, knitted, crocheted, felted, blown, etc.) to construct the dressing 300 subsequent the coating process. The uniform coating of the fiber substrate may be accomplished utilizing a metal-based coating process similar to the process 1900 of FIG. 19 without undue experimentation.

A similar process is used by Argentum Medical, LLC. of Chicago, Ill., for coating their “Silverlon®” antibacterial woven dressing product line. Although portions of the process are proprietary and may not be publicly known, similar techniques for metal-coating fiber, will be known to those skilled in the art.

While the foregoing description is exemplary of the preferred embodiments, those of ordinary skill in the relevant arts will recognize the many variations, alterations, modifications, substitutions and the like that are readily possible. It is contemplated that the components and additives for the polymer-based or metal-based coating solution may vary widely to accommodate the various substrate materials and agent(s) to be released. The coating can be formulated specific to coat thickness. It may be formulated to allow for various particle sizes. The coating may be formulated to provide various release kinetics, including but not limited to concentration, rate and the duration of agent release. For example, the release profile may be engineered such that release occurs in a matter of hours for up to several weeks. Concentration of delivery can be engineered to release from a low concentration of parts-per-billion (ppb) to several hundred parts-per-million (ppm) of agent within minutes. In the case where multiple agents are to be released, the coating may be formulated to provide scheduled and alternating agent releases.

Further, it is contemplated that the method of coating application or deposition may also vary widely, based on the various potential substrate materials and agent(s) to be released. The substrate material may vary beyond that set forth. Examples of the substrate useful in these embodiments include, but are not limited to foam, yarns, films, filaments, fibers, fabrics, filler materials, and a combination thereof that can be formed into the dressing 300.

It is also contemplated that the coating may incorporate single or multiple agents for release. Agents useful in these embodiments include, but are not limited to therapeutic and prophylactic agents, such as antimicrobial agents, enzymatic debriders, anesthetic agents, chemotherapeutic agents, indicating agents, and growth factors. Antimicrobial agents include, but are not limited to antibacterial agents, such as antibiotic and bacteriostatic agents. Useful indicating agents include, but are not limited to crystal violet, methylene blue, and similar agents known to cause a color change in tissue and/or body-liquid, for example, when in the presence of a bacterial agent, acidity, and alkalinity. Growth factors useful in embodiments discussed herein include, but are not limited to transforming growth factor, epidermal growth factor, platelet derived growth factor, insulin-like growth factor, keratinocyte growth factor, fibroblast growth factor, granulocyte macrophage colony stimulating factor, and granulocyte colony stimulating factor.

It is further contemplated that the screen 300 may comprise a plurality of portions, such as layers, only one of which comprises the uniformly covered substrate portion of the screen. In one embodiment, the screen 300 may be comprised of a lower uniformly covered substrate portion and an upper impermeable film portion of the screen, wherein the upper film portion of the screen may include an aperture or plurality of flow ports to provide fluid communication between the uniformly covered substrate portion of screen and the source of subatmospheric pressure. In an alternative embodiment, each of the plurality of portions of the screen may be comprised of substrate covered with different or alternating coatings for releasing a plurality of therapeutic or prophylactic agents to the tissue site 302.

While the foregoing description is exemplary of the preferred embodiments of the present invention, those of ordinary skill in the relevant arts will recognize the many other alternatives, variations, alterations, modifications, substitutions and the like as are readily possible, especially in light of this description, the accompanying drawings and claims drawn thereto. In any case, because the scope of the present invention is much broader than any particular embodiment, the foregoing detailed description should not be construed as a limitation of the scope of the present invention, which is limited only by the claims appended hereto.

We claim:

1. A system for treating tissue beneath a cover, the system comprising:
a screen placed in contact with the tissue;
a subatmospheric pressure tissue treatment device adapted to apply a subatmospheric pressure to the tissue, the subatmospheric pressure tissue treatment device comprising:
a source of subatmospheric pressure;
the cover positioned to enclose the screen and define a space under the cover and over the tissue;
a liquid conduit connected between the source of subatmospheric pressure and the space defined by the cover, wherein the liquid conduit provides a pathway to apply the subatmospheric pressure within the space defined by the cover and for drawing body-liquid therefrom;
a container connected to the liquid conduit between the source of subatmospheric pressure and the cover, wherein the container receives the body-liquid drawn through the liquid conduit from the space defined by the cover;
a filter interposed between the source of subatmospheric pressure and the container, wherein the filter prevents the body-liquid collected in the container from contacting the source of subatmospheric pressure;
a connector interposed between the liquid conduit and the space defined by the cover, wherein the connector secures the liquid conduit to the cover;
wherein at least a portion of the screen comprises a substrate uniformly covered with a coating;
wherein the uniformly covered substrate portion of the screen maintains physical properties of the substrate and enables severing of the screen in any direction to expose at least one coated surface of the uniformly covered substrate portion of the screen;
wherein the coating comprises at least silver and releases at least a portion of the at least silver within the space defined by the cover; and
wherein the subatmospheric pressure applied by the subatmospheric pressure tissue treatment device within the space defined by the cover is less than an ambient atmospheric pressure over the cover.

2. The system of claim 1, wherein the cover comprises an impermeable film having a pressure-sensitive adhesive coating thereon.

3. The system of claim 2, wherein the pressure-sensitive adhesive coating provides a substantially air-tight seal with a second region of tissue surrounding the tissue under the cover.

4. The system of claim 1, wherein the cover comprises a semi-rigid, impermeable cup having a cuff.

5. The system of claim 4, wherein the cuff provides a substantially air-tight seal about the tissue under the cover and holds the cup in place during application of the subatmospheric pressure within the space defined by the cover.

6. The system of claim 1, further comprising a pump pressure transducer connected to the liquid conduit between the source of subatmospheric pressure and the filter.

7. The system of claim 6, wherein the pump pressure transducer detects a pressure drop within the liquid conduit indicative of the body-liquid collected in the container substantially covering the filter.

8. The system of claim 1, further comprising a tissue pressure transducer fluidly connected to the space defined by the cover by a pressure detection conduit.

9. The system of claim 8, wherein the tissue pressure transducer measures a pressure within the space defined by the cover.

10. The system of claim 1, wherein a distal segment of the liquid conduit connected between the container and the space defined by the cover is longitudinally partitioned to provide a multi-lumen conduit comprising:
at least one liquid lumen connected between the container and the space defined by the cover, wherein the at least one liquid lumen provides the pathway for application of the subatmospheric pressure within the space defined by the cover, and
at least one pressure detection lumen connected between the container and the space defined by the cover, wherein the at least one pressure detection lumen is in fluid communication with the tissue pressure transducer and provides the pathway for measuring the pressure at or proximal the space defined by the cover.

11. The system of claim 10, wherein the at least one liquid lumen provides the pathway for drawing body-liquid from the space defined by the cover.

12. The system of claim 1, further comprising a relief valve connected to the space defined by the cover via the pressure detection conduit, wherein the relief valve admits air into the pressure detection conduit to bring the ambient atmospheric pressure to the space defined by the cover.

13. The system of claim 12, further comprising a controller connected to the relief valve and to the source of subatmospheric pressure, wherein the controller operates each accordingly, to intermittently apply the subatmospheric pressure to the space defined by the cover and bring the ambient atmospheric pressure to the space defined by the cover.

14. The system of claim 1, further comprising an access port connected to the liquid conduit for sampling the body-liquid drawn from within the spaced defined by the cover.

15. The system of claim 14, wherein the access port comprises a resealable membrane operable to maintain a seal after being punctured.

16. The system of claim 1, wherein the silver reduces bacterial density within the space defined by the cover.

17. The system of claim 1, wherein the substrate includes at least one material selected from a group including foam, yarn, film, filament, fiber, fabric, and filler material.

18. The system of claim 17, wherein the foam comprises polyurethane or polyvinylalcohol.

19. The system of claim 17, wherein the fiber includes at least one substance selected from the group including nylon, polyester, acrylic, rayon, cotton, polyurethane, other polymeric materials and cellulose.

20. The system of claim 1, further comprising an area of contact between the tissue and the uniformly covered substrate portion of the screen, wherein the area of contact increases as the screen conforms to the contacted tissue surface during application of the subatmospheric pressure within the space defined by the cover.

21. The system of claim 20, wherein the coating releases the at least silver to the increased area of contacted tissue.

22. The system of claim 21, wherein the at least silver reduces bacterial density on the increased area of contacted tissue.

23. The system of claim 1, wherein the screen further comprises a plurality of flow ports.

24. The system of claim 23, wherein the plurality of flow ports are uniformly covered with the coating.

25. The system of claim 1, wherein the coating is selected from a group including a metallic coating and a polymer coating.

26. The system of claim 25, wherein the substrate is uniformly covered with the coating utilizing an electroless reduction-oxidation process.

27. The system of claim 1, wherein the silver is in the form of metallic silver or silver salt powder.

28. The system of claim 1, wherein the coating includes at least one therapeutic or prophylactic agent selected from a group including antimicrobial agents, debriding agents, anesthetic agents, chemotherapeutic agents, indicating agents, and growth factors.

29. The system of claim 28, wherein the growth factor is at least one growth factor selected from a group including transforming growth factor, epidermal growth factor, platelet derived growth factor, insulin-like growth factor, keratinocyte growth factor, fibroblast growth factor, granulocyte macrophage colony stimulating factor, and granulocyte colony stimulating factor.

30. The system of claim 28, wherein the indicating agent indicates at least one condition of the tissue selected from a group of conditions including bacterial infection, acidity and alkalinity.

31. The system of claim 30, wherein the indicating agent is at least one indicating agent selected from a group including crystal violet and methylene blue.

32. The system of claim 1, wherein the tissue is at least one tissue selected from a group including bone tissue, adipose tissue, muscle tissue, dermal tissue, vascular tissue, connective tissue, cartilage, tendon, and ligament.

33. The system of claim 1, wherein the tissue is at least a portion of a wound.

34. The system of claim 33, wherein the wound is at least one wound selected from a group including partial thickness burns, traumatic wounds, surgical wounds, dehisced wounds, diabetic wounds, pressure ulcers, leg ulcers, flaps and grafts.

35. A system for treating tissue, the system comprising:
a cover defining a space between the cover and the tissue;
a screen placed in the space defined by the cover;
a source of subatmospheric pressure;
a pathway between the source of subatmospheric pressure and the space defined by the cover for application of a subatmospheric pressure within the space defined by the cover; and
wherein at least a portion of the screen is uniformly covered with a metallic silver coating.

36. The system of claim 35, wherein the metallic silver coating releases at least a portion of silver within the space defined by the cover when the coating contacts liquid drawn from the tissue.

37. The system of claim 36, wherein the silver reduces bacterial density within the space defined by the cover.

38. The system of claim 36, wherein the cover encloses the screen and extends beyond the edges of the screen.

39. The system of claim 36, further comprising a container connected to the pathway between the source of subatmospheric pressure and the space defined by the cover, wherein the container receives body-liquid drawn along the pathway from within the space defined by the cover.

40. The system of claim 39, wherein the silver reduces bacterial density within the container.

41. The system of claim 35, wherein the screen comprises a plurality of flow ports.

42. The system of claim 41, wherein the plurality of flow ports are uniformly covered with the metallic silver coating.

43. The system of claim 35, wherein the uniformly covered portion of the screen includes at least one material selected from a group including foam, yarn, film, filament, fiber, fabric, and filler material.

44. The system of claim 43, wherein the foam comprises polyurethane or polyvinylalcohol.

45. The system of claim 43, wherein the fiber includes at least one substance selected from the group including nylon, polyester, acrylic, rayon, cotton, polyurethane, other polymeric materials and cellulose.

46. The system of claim 35, wherein an area of contact between the tissue and the uniformly covered portion of the screen increases during application of the subatmospheric pressure.

47. The system of claim 46, wherein the metallic coating releases the silver to the increased area of contacted tissue.

48. The system of claim 47, wherein the silver reduces bacterial density on the increased area of contacted tissue.

49. The system of claim 35, wherein the tissue is at least one tissue selected from a group including bone tissue, adipose tissue, muscle tissue, dermal tissue, vascular tissue, connective tissue, cartilage, tendon, and ligament.

50. The system of claim 35, wherein the tissue is at least a portion of a wound selected from a group of wounds including partial thickness burns, traumatic wounds, surgical wounds, dehisced wounds, diabetic wounds, pressure ulcers, leg ulcers, flaps and grafts.

51. A method for treating tissue, the method comprising the steps of:
creating a coating solution comprising at least one therapeutic or prophylactic agent;
immersing a substrate in the coating solution;
uniformly covering the interior and exterior surfaces of the substrate with a coating comprising the at least one therapeutic or prophylactic agent;
removing excess coating solution from the uniformly covered substrate;
drying the uniformly covered substrate;
forming a screen, wherein at least a portion of the screen comprises the uniformly covered substrate;
severing the screen to fit a tissue site and exposing at least one coated surface of the uniformly covered substrate portion of the screen;
positioning the uniformly covered substrate portion of the screen in contact with the tissue;
enclosing the screen with a cover and defining a space under the cover and over the tissue;
connecting a liquid conduit between the space defined by the cover and a source of subatmospheric pressure to provide a pathway for applying a subatmospheric pressure within the space defined by the cover;
connecting a container to the liquid conduit between the source of subatmospheric pressure and the cover to receive the liquid drawn from the space defined by the cover;
increasing an area of contact between the tissue and the uniformly covered substrate portion of the screen by applying the subatmospheric pressure within the space defined by the cover;
contacting the increased area of contacted tissue with the coating comprising the at least one therapeutic or prophylactic agent; and
releasing at least a portion of the at least one therapeutic or prophylactic agent to the area of contacted tissue.

52. The method of claim 51, further comprising the step of detecting a pressure drop within the liquid conduit indicative of the container being full of the body-liquid.

53. The method of claim 52, further comprising the step of initiating an alarm indicative that the container is full of the body-liquid.

54. The method of claim 51, further comprising the step of measuring a pressure within the space defined by the cover.

55. The method of claim 51, further comprising the step of bringing ambient atmospheric pressure to the space defined by the cover.

56. The system of claim 51, further comprising the steps of intermittently applying the subatmospheric pressure to the space defined by the cover and bringing the ambient atmospheric pressure to the space defined by the cover.

57. The method of claim 51, further comprising the step of sampling the body-liquid within the liquid conduit while applying the subatmospheric pressure to the space defined by the cover.

58. The method of claim 51, wherein the at least one therapeutic or prophylactic agent is silver.

59. The method of claim 58, further comprising the step of reducing bacterial density within the space defined by the cover.

60. The method of claim 58 further comprising the step of reducing bacterial density within the container.

61. The method of claim 51, wherein the subatmospheric pressure is less than ambient atmospheric pressure surrounding the cover.

62. The method of claim 51, wherein the substrate includes at least one material selected from a group including foam, yarn, film, filament, fiber, fabric, and filler material.

63. The method of claim 62, wherein the foam comprises polyurethane or polyvinylalcohol.

64. The method of claim 62, wherein the fiber includes at least one substance selected from the group including nylon, polyester, acrylic, rayon, cotton, polyurethane, other polymeric materials and cellulose.

65. The method of claim 51, wherein the coating solution is selected from a group including a metallic coating solution and a polymer coating solution.

66. The method of claim 65, wherein the step of uniformly covering the substrate with the coating utilizes an electroless reduction-oxidation process.

67. The method of claim 51, wherein the at least one therapeutic or prophylactic agent is selected from a group including antimicrobial agents, debriding agents, anesthetic agents, chemotherapeutic agents, indicating agents, and growth factors.

68. The method of claim 67, wherein the growth factor is at least one growth factor selected from a group including transforming growth factor, epidermal growth factor, platelet derived growth factor, insulin-like growth factor, keratinocyte growth factor, fibroblast growth factor, granulocyte macrophage colony stimulating factor, and granulocyte colony stimulating factor.

69. The method of claim 67, wherein the indicating agent is at least one indicating agent selected from a group including crystal violet and methylene blue.

70. The method of claim 69, further comprising the step of indicating at least one condition of the tissue selected from a group of conditions including bacterial infection, acidity, and alkalinity.

71. The method of claim 51, wherein the tissue is at least one tissue selected from a group including bone tissue, adipose tissue, muscle tissue, dermal tissue, vascular tissue, connective tissue, cartilage, tendon, and ligament.

72. The method of claim 51, wherein the tissue is at least a portion of a wound.

73. The method of claim 72, wherein the wound is at least one wound selected from a group including partial thickness burns, traumatic wounds, surgical wounds, dehisced wounds, diabetic wounds, pressure ulcers, leg ulcers, flaps and grafts.

74. A method for treating tissue beneath a cover, the method comprising the steps of:
forming a screen, wherein the screen comprises at least a substrate uniformly covered with a coating comprising at least one therapeutic or prophylactic agent, and wherein at least one coated surface of the uniformly covered substrate portion of the screen is exposed when the screen is severed for fitting a tissue site;
positioning the uniformly covered substrate portion of the screen in contact with the tissue;
placing the cover over the screen to define a space between the cover and the tissue;
connecting a liquid conduit between the space defined by the cover and a source of subatmospheric pressure to provide a pathway for applying a subatmospheric pressure within the space defined by the cover;
increasing an area of contact between the tissue and the uniformly covered substrate portion of the screen by applying the subatmospheric pressure within the space defined by the cover; and
releasing at least a portion of the at least one therapeutic or prophylactic agent to the area of contacted tissue.

75. The method of claim 74, wherein the liquid conduit further provides a pathway for drawing body-liquid from the space defined by the cover into a container.

76. The method of claim 75, further comprising the step of detecting a pressure drop within the liquid conduit indicative of the container being full of the body-liquid.

77. The method of claim 76, further comprising the step of initiating an alarm indicative that the container is full of the body-liquid.

78. The method of claim 74, further comprising the step of measuring a pressure within the space defined by the cover.

79. The method of claim 74, further comprising the step of bringing ambient atmospheric pressure to the space defined by the cover.

80. The method of claim 74, further comprising the steps of intermittently applying the subatmospheric pressure to the space defined by the cover and bringing the ambient atmospheric pressure to the space defined by the cover.

81. The method of claim 74, wherein the subatmospheric pressure is less than an ambient atmospheric pressure surrounding the cover.

82. The method of claim 74, wherein the substrate includes at least one material selected from a group including foam, yarn, film, filament, fiber, fabric, and filler material.

83. The method of claim 82, wherein the foam comprises polyurethane or polyvinylalcohol.

84. The method of claim 82, wherein the fiber includes at least one substance selected from the group including nylon, polyester, acrylic, rayon, cotton, polyurethane, other polymeric materials and cellulose.

85. The method of claim 74, wherein the at least one therapeutic or prophylactic agent is selected from a group including antimicrobial agents, debriding agents, anesthetic agents, chemotherapeutic agents, indicating agents, and growth factors.

86. The method of claim 85, wherein the at least one therapeutic or prophylactic agent is silver.

87. The method of claim 75, further comprising the step of reducing bacterial density within the space defined by the cover.

88. The method of claim 86, further comprising the step of reducing bacterial density within the container.

89. The method of claim 85, wherein the growth factor is at least one growth factor selected from a group including transforming growth factor, epidermal growth factor, platelet derived growth factor, insulin-like growth factor, keratinocyte growth factor, fibroblast growth factor, granulocyte macrophage colony stimulating factor, and granulocyte colony stimulating factor.

90. The method of claim 85, wherein the indicating agent is at least one indicating agent selected from a group including crystal violet and methylene blue.

91. The method of claim 90, further comprising the step of indicating at least one condition of the tissue selected from a group of conditions including bacterial infection, acidity, and alkalinity.

92. The method of claim 74, wherein the tissue is at least one tissue selected from a group including bone tissue, adipose tissue, muscle tissue, dermal tissue, vascular tissue, connective tissue, cartilage, tendon, and ligament.

93. The method of claim 74, wherein the tissue is at least a portion of a wound.

94. The method of claim 93, wherein the wound is at least one wound selected from a group including partial thickness burns, traumatic wounds, surgical wounds, dehisced wounds, diabetic wounds, pressure ulcers, leg ulcers, flaps and grafts.

95. A method for adapting a dressing for delivering an agent to a wound site during application of subatmospheric pressure tissue treatment, the method comprising the steps of:
creating a coating solution comprising an antimicrobial agent;
immersing the dressing in the coating solution;
uniformly coating the dressing with a coating comprising the antimicrobial agent, wherein an upper surface, a lower surface, side surfaces, and interior surfaces of the dressing are uniformly coated;
removing excess coating solution from a uniformly coated dressing;
drying the uniformly coated dressing; and
severing the uniformly coated dressing as needed to match a size and shape of the wound site, wherein all exposed surfaces of the severed dressing are uniformly coated sufficient to treat the wound site during application of a subatmospheric pressure.

96. The method of claim 95, wherein the dressing comprises foam.

97. The method of claim 96, wherein the foam comprises polyurethane.

98. The method of claim 95, wherein the antimicrobial agent is silver.
